(19) United States
(12) Patent Application Publication
(10) Pub. No.: US 2014/0234424 A1
(43) Pub. Date: Aug. 21, 2014

(54) PRAME PURIFICATION

(71) Applicants: Olivier C Germay, Rixensart (BE); Stephane Andre Godart, Rixensart (BE); Pol Guy Harvengt, Rixensart (BE); Amina Laanan, Rixensart (BE); Olivier Patrick Le Bussy, Rixensart (BE); Dominique Ingrid Lemoine, Rixensart (BE); Leonard Dode, Rixensart (BE)

(72) Inventors: Olivier C Germay, Rixensart (BE); Stephane Andre Godart, Rixensart (BE); Pol Guy Harvengt, Rixensart (BE); Amina Laanan, Rixensart (BE); Olivier Patrick Le Bussy, Rixensart (BE); Dominique Ingrid Lemoine, Rixensart (BE); Leonard Dode, Rixensart (BE)

(73) Assignee: GLAXOSMITHKLINE BIOLOGICALS SA, Rixensart (BE)

(21) Appl. No.: 14/234,111

(22) PCT Filed: Jul. 20, 2012
(86) PCT No.: PCT/EP2012/064340

§ 371 Date: Jan. 21, 2014

(30) Foreign Application Priority Data
Jul. 22, 2011 (GB) 1112658.8
Sep. 12, 2011 (GB) 1115737.7

(51) Int. Cl. A61K 39/00; A61K 9/19

(52) U.S. Cl. CPC A61K 39/0011; A61K 9/19
USPC 424/489; 424/277.1; 530/350

(57) ABSTRACT

Methods and processes for the purification of PRAME are provided. In particular, methods for reducing the aggregation of PRAME during a diluent exchange from diluent A to diluent B comprising: (i) adding a polyanionic compound to diluent A prior to or contemporaneously with the exchange; and (ii) exchanging protein from diluent A to diluent B are provided. Compositions produced by the method are also provided.

### TECHNICAL FIELD

The present invention relates to methods for the purification of PRAME.

### BACKGROUND

PReferentially expressed Antigen in MElanoma”, or “PRAME”, is a tumour antigen encoded by the PRAME gene.

PRAME is an antigen that is over-expressed in many types of tumours, including melanoma, lung cancer and leukaemia (Ikeda et al., Immunity 1997, 6 (2) 199-208). A high level of PRAME expression has been reported for several solid tumors, including ovarian cancer, breast cancer, lung cancer and melanomas, medulloblastoma, sarcomas, head and neck cancers, neuroblastoma, renal cancer, and Wilms' tumour and in hematologic malignancies including acute lymphoblastic and myelogenous leukemias (ALL and AML), chronic myelogenous leukemia (CML), Hodgkin's disease, multiple myeloma, chronic lymphocytic leukemia (CLL) and mantle cell lymphoma (MCL).

PRAME is also expressed at a very low level in a few normal tissues, for example testis, adrenals, ovary and endometrium.

PRAME represents an important anti-cancer immunotherapeutic. In immunotherapy the cancer antigen is introduced to the patient usually as a vaccine, for example containing the protein or an antigenic fragment thereof, which stimulates the patient's immune system to kill tumours expressing the same antigen.

The production of a vaccine comprising the cancer antigen, in this case PRAME, requires a significant quantity of the cancer antigen, which in turn calls for the large scale expression and purification of the antigen.

### SUMMARY OF THE INVENTION

PRAME is over expressed in E. coli where it forms inclusion bodies. In order to solubilise PRAME from the inclusion bodies they must be exposed to strongly solubilising conditions requiring anionic detergent and urea. However, such conditions are not suitable for the final formulation of PRAME into a composition for injection into patients and the purified PRAME must be transferred to another diluent.

The inventors of the present application have realised that transfer of PRAME from a diluent comprising the anionic detergent used to solubilise it to one which is substantially free of that anionic detergent causes aggregation of PRAME. This aggregation continues over time and eventually causes precipitation of the PRAME out of solution. As this aggregation (antigen size evolution) is not suitable for use in an immunotherapeutic composition there is therefore a need in the art for improved methods for the purification of PRAME.

Methods and processes for reducing the aggregation of PRAME, as well as compounds produced by these methods and processes, are provided herein. In one embodiment there is provided a method for reducing the aggregation of a protein during a diluent exchange from diluent A to diluent B comprising: (i) adding a polyanionic compound to diluent A prior to the exchange; and (ii) exchanging the protein from diluent A to diluent B, wherein the protein is PRAME. In one embodiment there is provided the use of a polyanionic composition for reducing the aggregation of a protein during a diluent exchange from diluent A to diluent B, wherein the protein is PRAME. In one embodiment the polyanionic compound is added prior to the diluent exchange. In one embodiment diluent A comprises a detergent. In another embodiment the detergent is an anionic detergent. In another embodiment the detergent is selected from the group consisting of: SDS, sodium docusate and lauryl sarcosyl.

In one embodiment diluent B is substantially free of detergent.

In one embodiment the polyanionic compound is an oligonucleotide. In one embodiment the oligonucleotide is 5 to 200 nucleotides in length. In one embodiment, the oligonucleotide comprises a CpG. Most In one embodiment the oligonucleotide is selected from the group consisting of: TCC ATG ACG TTC CTG ACG TT (CpG 1826) (SEQ ID NO:1); TCT CCC AGC GTG CGC CAT (CpG 1758) (SEQ ID NO:2); ACC GAT GAC GTC GCC GGT GAC GGC ACC ACG (SEQ ID NO:3); TCG TCG TTT TGT CGT TTT GTC GTT (CpG 2006/CpG7909) (SEQ ID NO:4); TCC ATG ACG TTC CTG ATG CT (CpG 1668) (SEQ ID NO:5); or TCG ACG TTT TCG GCG CGC GCC G (CpG 5456) (SEQ ID NO:6).

In one embodiment, the diluent exchange is achieved by dialysis, diafiltration or size exclusion chromatography.

In one embodiment, the method further comprises step (iii) formulating the protein into diluent C. In one embodiment diluent C comprises Tris, Borate, sucrose, poloxamer and CpG.

The invention also provides a composition comprising PRAME in diluent C as produced by the methods of the invention.

The invention also provides a composition comprising PRAME and an oligonucleotide, wherein the PRAME has a particle size of between 10-30 nm. In another embodiment PRAME has a particle size of between 15-25 nm. In another embodiment, the oligonucleotide comprises a CpG. In a further embodiment, the particle size is determined by dynamic light scattering.

The invention also provides a method of producing a pharmaceutically acceptable PRAME composition comprising the steps of: (a) carrying out a diluent exchange according to the methods of the invention; (b) formulating the protein into diluent C; and (c) sterilising the formulation produced in step (b). In another embodiment, the method comprises the additional step (d) lyophilising the formulation produced in step (c). In another embodiment, step (c) is achieved by filtration.

### BRIEF DESCRIPTION OF THE FIGURES

FIG. 1/21: Electrophoretic mobility measurement and Zeta potential calculation for PRAME purified antigen with Malvern ZetaSizer Nano ZS equipment.

FIG. 2/21: Light scattering (LS), refractive index (RI) and molar mass (MM) distributions as determined by SEC-MALLS analyses for GMP lot DPRAAPA003 at release.

FIG. 3/21: Light scattering (LS), refractive index (RI) and molar mass (MM) distributions as determined by SEC-MALLS analyses for GMP lot DPRAAPA004 at release.

FIG. 4/21: Light scattering (LS), refractive index (RI) and molar mass (MM) distributions as determined by SEC-MALLS analyses for GMP lot DPRAAPA005 at release.

FIG. 5/21: Sedimentation coefficient distributions c(s) obtained by SV-AUC analyses of GMP lots DPRAAPA003 (blue profile), DPRAAPA004 (red profile) and DPRAAPA005 (green profile) at release. Note that the raw data obtained by SV-AUC have been processed using the Sedfit software. The waves are due to this signal treatment and, thus, are artifactual.

FIG. 6/21: SDS-PAGE analysis in reducing conditions on 4-12% Bis-Tris polyacrylamide gel Coomassie Blue R250 staining (5 μg of protein loaded per lane)-Final Container reconstituted in ASA (Sorbitol)-Follow-up of the reconstitution kinetic at 25° C. Lanes are numbered from left to right

FIG. 7/21: Western Blot analysis against PRAME antigen. Final Container reconstituted in ASA (Sorbitol) buffer or water. Follow-up of the reconstitution kinetic at 25° C. 0.3 μg of protein loaded per lane, transfer on nitrocellulose membrane 1 h at 100V, alkaline phosphatase (NBT-BCIP) detection. Lane 1: Final container (FC) reconstituted in water for injection at T0-non centrifuged sample; lane 2: idem 1-centrifuged sample (supernatant); lane 3: Final container (FC) reconstituted in ASA buffer at T0-non centrifuged sample; lane 4: idem 3-centrifuged sample (supernatant); lane 5: Final container (FC) reconstituted in ASA buffer at T 4 h 25° C.-non centrifuged sample; lane 6: idem 5-centrifuged sample (supernatant); lane 7: Final container (FC) reconstituted in ASA buffer at T24 h 25° C. -non centrifuged sample; lane 8: idem 7-centrifuged sample (supernatant).

FIG. 8/21: Isothermal titration calorimetry profile corresponding to the stepwise injection of CpG7909 into a PRAME solution. Binding of CpG to PRAME results in the characteristic sequence of the signal, until saturation is reached.

FIG. 9/21: Top panel represents the PRAME protein distribution visualized after silver staining of a SDS-PAGE gel. Bottom panel represents the CpG distribution along the gradient after IEX-HPLC-UV determination. Fraction 1 is equivalent to the bottom fraction highlighted above the corresponding lane of the SDS-PAGE gel. Similarly, fraction 12 is equivalent to the top fraction and fraction w is equivalent to the tube wash lane. Red box is meant to delineate the fractions were CpG is interacting with the antigen (in control experiment, CpG alone is found in top fractions only).

FIG. 10/21: Comparative data showing the amount of CpG associated with PRAME antigen for three distinct repro lots. Blue bars correspond to ex-tempo reconstitution of lyophilized materials (500 μg dose on left half of graph, 100 μg dose for right half). Green bars correspond to samples pre-incubated for 24 h at 25° C. before ultracentrifugation. Diamond-shaped in magenta correspond to the mass ratio CpG/Ag and should be read from the right axis.

FIG. 11/21: SEC-HPLC method development. SEC Column selection. UV profiles obtained on different TSK columns for purified antigen.

FIG. 12/21: SEC-HPLC method development. SEC Column selection. UV profiles obtained on different TSK columns for purified antigen spiked with CpG solution (1050 μg/ml).

FIG. 13/21: SEC-HPLC analysis on TSK G4000 PWxI+G6000 PWxI columns (+guard column) equilibrated in 5 mM Borate buffer pH 9.8-3.15% sucrose (=buffer of the purified antigen) at a flow-rate of 0.5 ml/min and with UV detection at 220 nm-UV profiles obtained for purified antigen alone or after spiking with increasing concentrations of CpG. CpG impact on antigen chromatographic profile. N.B. Vo=void volume of the column, i.e. the volume outside of the resin beads.

FIG. 14/21: SEC-HPLC analysis on TSK G4000 PWxI+G6000 PWxI columns (+guard column) equilibrated in 5 mM Borate buffer pH 9.8-3.15% sucrose (=buffer of the purified antigen) at a flow-rate of 0.5 ml/min and with UV detection at 220 nm-UV profiles obtained for CpG solution in water from 10 μg/ml up to 1050 μg/ml.

FIG. 15/21: Size analysis by dynamic light scattering (ZetaNano® from Malvern) on purified antigen samples spiked or not with excipient and stored 24 h at 22° C. (no size measurement done when antigen precipitation observed by visual observation).

FIG. 16/21: Size analysis by dynamic light scattering (ZetaNano® from Malvern) on purified antigen samples spiked with selected excipient candidates and stored 14 days at +4° C.

FIG. 17/21: Turbidity measurement (HACH 2100AN IS®) on purified antigen samples spiked with selected excipient candidates after 14 days at +4° C.

FIG. 18/21: Compatibility of ASA (Sorbitol) with ionic detergents—Size analysis by dynamic light scattering (ZetaNano® from Malvern).

FIG. 19/21: A graphical representation of DLS measurements.

FIG. 20/21: Visual analysis of a samples with no CpG (R19/1) and spiking with 100 μg/ml CpG in HA-FT prior UF (Run R26/1).

FIG. 21/21: A graphical representation of DLS measurements.

### DETAILED DESCRIPTION

The inventors have surprisingly found that the addition of a polyanionic compound to a diluent containing PRAME prior to the exchange of the diluent reduces the aggregation of PRAME.

### Aggregation

As discussed above, the methods of the invention reduce the aggregation of PRAME during a diluent exchange. Aggregation refers to the associating of individual PRAME molecules with other PRAME molecules to form multimers. Aggregation can be observed visually or using dynamic light scattering techniques well known in the art.

### PRAME

As described above, PRAME is an antigen that is over-expressed in many types of tumours, including melanoma, lung cancer and leukaemia (Ikeda et al., Immunity 1997, 6 (2) 199-208). The PRAME protein has 509 amino acids (SEQ ID NO:7). The antigen is described in U.S. Pat. No. 5,830,753. PRAME is also found in the Annotated Human Gene Database H-Inv DB under the accession numbers: U65011.1, BC0220081, AK129783.1, BC014974.2, CR608334.1, AF025440.1, CR591755.1, BC039731.1, CR623010.1, CR611321.1, CR618501.1, CR604772.1, CR456549.1, and CR620272.1. As used herein, the term PRAME includes the full length wild type PRAME protein. It also includes PRAME proteins with conservative substitutions. In one embodiment, one or more amino acids may be substituted, i.e. 2, 3, 4, 5, 6, 7, 8, 9, 10, 11, 12, 13, 14, 15, 16, 17, 18, 19 or more. The PRAME protein may additionally or alternatively contain deletions or insertions within the amino acid sequence when compared to the wild-type PRAME sequence. In one embodiment, one or more amino acids may be inserted or deleted, i.e. 2, 3, 4, 5, 6, 7, 8, 9, 10, 11, 12, 13, 14, 15, 16, 17, 18, 19 or more.

In one embodiment, the term PRAME includes proteins which share 80% or more sequence identity with the full length wild type PRAME protein, i.e., 85%, 90%, 95%, 96%, 97%, 98%, 99% or more.

The term PRAME also includes fusion protein proteins comprising the PRAME protein. PRAME may be fused or conjugated to a fusion partner or carrier protein. For example, the fusion partner or carrier protein may be selected from protein D, NS1 or CLytA or fragments thereof. See, e.g., WO2008/087102.

In one embodiment of the invention, the immunological fusion partner that may be used is derived from protein D, a surface protein of the gram-negative bacterium, Haemophilus influenza B (WO91/18926) or a derivative thereof. The protein D derivative may comprise the first ⅓ of the protein, or approximately the first ⅓ of the protein. In one embodiment, the first 109 residues of protein D may be used as a fusion partner. In an alternative embodiment, the protein D derivative may comprise the first N-terminal 100-110 amino acids or about or approximately the first N-terminal 100-110 amino acids. In one embodiment, the protein D or derivative thereof may be lipidated and lipoprotein D may be used.

In one embodiment, the PRAME protein is a fusion protein comprising: a) PRAME or an immunogenic fragment thereof, and b) a heterologous fusion partner derived from protein D, wherein the said fusion protein does not include the secretion sequence (signal sequence) of protein D. By secretion or signal sequence or secretion signal of protein D is meant the N-terminal 19 amino acids of protein D. Thus, the fusion partner protein of the present invention may comprise the remaining full length protein D protein, or may comprise approximately the remaining N-terminal third of protein D. For example, the remaining N-terminal third of protein D may comprise approximately or about amino acids 20 to 127 of protein D. In one embodiment, the protein D sequence comprises N-terminal amino acids 20 to 127 of protein D.

In one embodiment, the PRAME may be Protein D-PRAME/His, a fusion protein comprising from N-terminal to C-terminal: amino acids Met-Asp-Pro; amino acids 20 to 127 of Protein D; PRAME; an optional linker; and a polyhistidine tail (His). Examples of linkers and polyhistidine tails that may optionally be used include for example: TSGHHHHHH; LEHHHHHH or HHHHHH.

PRAME as used in the present invention will usually be at a concentration between 10-2000 mg/ml, i.e. 2, 5, 10, 15, 20, 25, 30, 35, 40, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1750 or 2000 mg/ml.

### Polyelectrolytes and Polyanionic Compounds

Polyelectrolytes are polymers whose repeating units bear an electrolyte group. These groups will dissociate in aqueous solutions (water), making the polymers charged. Polyelectrolyte properties are thus similar to both electrolytes (salts) and polymers (high molecular weight compounds), and are sometimes called polysalts. Like salts, their solutions are electrically conductive. Like polymers, their solutions are often viscous.

As referred to herein, a polyanionic compound is a polyelectrolyte with an overall negative charge. Examples of polyanionic compounds include, but are not limited to, PLG and oligonucleotides.

The net negative charge at pH7.0 of the polyanionic compound may be calculated by any suitable means. This may be an average property of the compound, and should be calculated with respect to the Mw of the polyanionic compound used. For instance, a PLG polymer with on average 17 residues should have a net negative charge of 17. In one embodiment, the net negative charge should be at least 8, or at least 17, preferably between 8-100, 10-80, 12-60, 14-40, 16-20, and most preferably about or exactly 17.

In one embodiment the polyanionic compound of the invention has at least one average 1 net negative charge at pH 7.0 per 3 monomers, preferably at least 2 per 3 monomers, and most preferably at least on average 1 net negative charge for each 30 monomer. The charges may be unevenly arranged over the compound length, but are preferably evenly spread over the compound length.

The skilled person will appreciate that the term polyanionic compound may include polyanionic detergents. However where the invention refers to adding a polyanionic compound to diluent A prior to a diluent exchange from diluent A to diluent B, wherein diluent A comprises an anionic detergent, then the anionic detergent is not the same as the polyanionic compound added to diluent A.

### Poly L-Glutamate (PLG)

Poly L-glutamate is a polymer of 1-glutamate used to stabilise diluents comprising biological molecules. In one embodiment, low molecular weight PLG (less than 6000 Mw, preferably 640-5000) is used (for instance PLG with on average 17 residues with a Mw of 2178). PLG is a fully bio-degradable polyamino acid with a pendent free y-carboxyl group in each repeat unit (pKa 4.1) and is negatively charged at a pH7, which renders this homopolymer water-soluble and gives it a polyanionic structure. PLG may be made using conventional peptide synthesis techniques. It is also available from Sigma-Aldrich, St. Louis, Mo., USA, in a relatively polydisperse form (e.g. 17mers with a polydispersity around 2.6), or from Neosystem, Strasbourg, France in a relatively monodisperse form (e.g. 8, 16, 24 or 32mers with a polydispersity close to 1).

PLG as used in the present invention will usually be at a concentration between 10-2000 μg/ml, i.e. 2, 5, 10, 15, 20, 25, 30, 35, 40, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1750 or 2000 μg/ml.

### Oligonucleotide

The oligonucleotides for use in the present invention may be composed of ribonucleic acid, deoxyribonucleic acid or any chemically modified nucleic acid known in the art. However, the oligonucleotides utilised in the present invention are typically deoxynucleotides. The oligonucleotides may contain any sequence of purines or pyrimidines.

In one embodiment the oligonucleotide comprises a CpG. CpG is an abbreviation for cytosine-guanosine dinucleotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the dinucleotide CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequence containing oligonucleotides can activate various immune subsets, including natural killer cells (which produce interferon γ and have cytolytic activity) and macrophages (Wooldrige et al Vol 89 (no. 8), 1977). Although other unmethylated CpG containing sequences not having this consensus sequence have now been shown to be immunomodulatory.

In one embodiment of the invention, the oligonucleotide contains two or more dinucleotide CpG motifs separated by at least three, preferably at least six or more nucleotides. The oligonucleotides of the present invention are typically deoxynucleotides. In a preferred embodiment the internucleotide bond in the oligonucleotide is phosphorodithioate, or more preferably a phosphorothioate bond, although phosphodiester and other internucleotide bonds are within the scope of the invention including oligonucleotides with mixed internucleotide linkages. Examples of preferred oligonucleotides have the following sequences. The sequences preferably contain phosphorothioate modified internucleotide linkages.

SEQ ID NO: 1
TCC ATG ACG TTC CTG ACG TT (CpG 1826;) -
SEQ ID NO: 2
TCT CCC AGC GTG CGC CAT (CpG 1758) -
SEQ ID NO: 3
ACC GAT GAC GTC GCC GGT GAC GGC ACC ACG -
SEQ ID NO: 4
TCG TCG TTT TGT CGT TTT GTC GTT
(CpG 2006/CpG7909)
SEQ ID NO: 5
TCC ATG ACG TTC CTG ATG CT
(CpG 1668)
SEQ ID NO: 6
TCG ACG TTT TCG GCG CGC GCC G
(CpG 5456)
SEQ ID NO: 9
TCG TCG TTT TGT CGT
(CpG 15 mer):
SEQ ID NO: 10
TCG TCG TTT TGT CGT TTT GTC GTT TCG TCG
(CpG 30 mer):

Alternative CpG oligonucleotides may comprise the preferred sequences above in that they have inconsequential deletions or additions thereto.

The CpG oligonucleotides utilised in the present invention may be synthesized by any method known in the art (eg EP 468520). Conveniently, such oligonucleotides may be synthesized utilising an automated synthesizer.

Oligonucleotides for use in the present invention are usually 2-500 bases in length, i.e. 2, 5, 10, 15, 20, 25, 30, 35, 40, 50, 75, 100, 125, 150, 175, 200, 250, 300, 350, 400, 450, or 500 bases. In one embodiment the oligonucleotides for use in the present invention are 10-50 bases in length, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length.

Oligonucleotides as used in the present invention will usually be at a concentration between 10-2000 μg/ml, i.e. 2, 5, 10, 15, 20, 25, 30, 35, 40, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1750 or 2000 μg/ml.

### Diluent

The term diluent refers to a diluting agent. In the context of the present invention the diluent may refer to the diluent alone, or it may refer to the diluent comprising one or more solutes. These solutes can be any molecule, including, but not limited to salts, buffers, detergents, polymers, proteins and/or oligonucleotides. The diluent will usually be water, but may also be another suitable solvent.

### Diluent A

As described above, as PRAME is over expressed in E. coli, where it forms inclusion bodies, in order to solubilise PRAME it is necessary to expose the inclusion bodies to strongly solubilising conditions requiring anionic detergent and urea. It is also necessary to keep PRAME soluble during the purification process. Diluent A may refer to the diluent which is used to directly solubilise PRAME from the cells in which it is expressed or it may refer to any buffer used during the purification of PRAME. The term “Diluent A” can be used to refer to the diluent irrespective of the presence of the polyanionic compound. As referred to herein, diluent A is any diluent used in the presently disclosed process for the purification of PRAME.

In one embodiment, diluent A will usually comprise a detergent. In one embodiment, the detergent will usually be at a concentration less than 0.1% w/v. In a further embodiment the detergent will be an anionic detergent. An anionic detergent is any detergent in which the lipophilic part of the molecule is an anion; examples include soaps and synthetic long-chain sulfates and sulfonates. In one embodiment the anionic detergent is sodium dodecyl sulphate (SDS), sodium docusate or lauryl sarcosyl.

In one embodiment, diluent A comprises one or more of Tris, NaH₂PO₄.2H₂O, urea and lauryl sarcosyl.

Where present the Tris will be at a concentration between 1-200 mM, i.e. 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175 or 200 mM.

Where present the NaH₂PO₄.2H₂O will be at a concentration between 1-200 mM, i.e. 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 mM.

Where present the Urea will be at a concentration between 0.5-9M, i.e. 0.5, 1.0, 1.5, 2.0, 2.5, 3.0, 3.5, 4.0, 4.5, 5.0, 5.5, 6.0, 6.5, 7.0, 7.5, 8.0, 8.5 or 9.0M.

Where present the lauryl sarcosyl will be at a concentration between 0.1-10% w/v, i.e. 0.1, 0.2, 0.3, 0.4, 0.5, 0.6, 0.7, 0.8, 0.9, 1, 2, 3, 4, 5, 6, 7, 8, 9, or 10% w/v.

### Diluent B

As described above, in order to use PRAME in a composition for injection in patients, it must be transferred to a suitable diluent. Such a diluent will usually be substantially free of the detergents used in the solubilisation and purification of PRAME. In one embodiment diluent B will be substantially free of detergent.

The term “substantially free” means that there will be less than 0.1% w/v of detergent, i.e. 0.09, 0.08, 0.07, 0.06, 0.05, 0.04, 0.03, 0.02, 0.01% or less w/v of detergent. In a further embodiment the term “substantially free” means that there will be less than 0.01% w/v detergent. i.e. 0.009, 0.008, 0.007, 0.006, 0.005, 0.004, 0.003, 0.002, 0.001%, 0.0005% or less w/v of detergent.

In one embodiment, diluent B comprises one or more of Borate and sucrose. In one embodiment, diluent B comprises Borate and sucrose.

Where present the borate will be at a concentration between 1-200 mM, i.e. 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 mM.

Where present the sucrose will be at a concentration between 0.1-20% w/v, i.e. 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20% w/v.

### Diluent C

Once the PRAME has been exchanged from diluent A to B, it may be necessary to formulate PRAME into a new diluent, diluent C. For example, diluent C may be used to store PRAME, may be to allow lyophilisation of PRAME, or may be for direct use in a patient.

In order to formulate PRAME in to diluent C, PRAME containing diluent B may undergo diluent exchange with diluent C using the processes described above. Additional components may be added to the PRAME containing diluent B in order to arrive at a new diluent, diluent C. In addition or alternatively, diluent B may be diluted to arrive at diluent C. All of these methods are contemplated by the invention.

In one embodiment, diluent C comprises one or more of Tris, borate, sucrose, poloaxmer and CpG. In one embodiment, diluent C comprises Tris, borate, sucrose, poloaxmer and CpG.

Where present the Tris will be at a concentration between 1-200 mM, i.e. 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175 or 200 mM.

Where present the borate will be at a concentration between 1-200 mM, i.e. 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 mM.

Where present the poloxamer will be at a concentration between 0.01-2% w/v, i.e. 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.60, 0.70, 0.80, 0.90, 1.0, 1.25, 1.50, 1.75, or 2% w/v. In one embodiment the poloxamer is poloxamer 188.

Where present the sucrose will be at a concentration between 0.1-20% w/v, i.e. 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20% w/v.

Where present, the CpG will be at a concentration between 10-2000 μg/ml, i.e. 2, 5, 10, 15, 20, 25, 30, 35, 40, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1750 or 2000 μg/ml.

Diluent C may be at a pH in the range of 5-10, i.e. a pH of 5, 5.5, 6.0, 6.5, 7.0, 7.5, 8.0, 8.5, 8.6, 8.7, 8.8, 8.9, 9.0, 9.1, 9.2, 9.3, 9.4, 9.5, 9.6, 9.7, 9.8 9.9 or 10.

### Diluent Exchange

Diluent exchange refers to the transfer of protein from a first diluent to a second diluent. The protein may itself be transferred, but it is more common for the diluent to be transferred. Examples of diluent exchange include, but are not limited to, dialysis, Diafiltration and size exclusion chromatography.

As described herein, the aim of the invention is to reduce the aggregation of a protein during a diluent exchange. The methods of the invention refer to adding a polyanionic compound to diluent A prior to diluent exchange with diluent B. The skilled person will appreciate, however, that there are situations where the polyanionic compound can be added to diluent A contemporaneously with the diluent exchange. For example, the polyanionic compound may be present in diluent B. Upon commencement of the diluent exchange, polyanionic compound present in diluent B will be added to diluent A. In another example, the polyanionic compound may be added to a combination of diluent A and B after the diluent exchange has begun. Such situations are also contemplated by the invention.

### Dialysis

Dialysis relies on the separation of particles in a liquid on the basis of differences in their ability to pass through a membrane. For example, a small volume of diluent A containing a protein is placed into a semi-permeable membrane which is sealed. The membrane is then placed into a larger volume of a diluent B. The membrane allows the movement of small solute molecules and solvent across the semi-permeable membrane, but not the larger protein molecules. After a period of time, the diluent on the outside and inside of the membrane equilibrates. Because of the large difference in volume of the two diluents, equilibration effectively results in the replacement of diluent A with diluent B.

### Diafiltration

Diafiltration is also a membrane based separation that is used to exchange diluents. In batch diafiltration, diluent A is typically diluted by a factor of two using new diluent, i.e. diluent B, brought back to the original volume by tangential flow filtration (TFF), permeate elimination is used to reduce the volume to initial value, and the whole process repeated several times to achieve the elimination of original diluent A. In continuous diafiltration the diluent B is added at the same rate as the permeate flow.

### Compositions

As described above, the problem identified and solved by the inventors of the present application is related to the aggregation of PRAME. Transfer of PRAME from a diluent comprising a strong detergent to one which is substantially free of detergent causes the aggregation of PRAME. This aggregation continues over time and eventually causes precipitation of the PRAME out of solution.

The methods of the invention described above solve this problem and allow the production of PRAME composition which has a consistent hydrodynamic radius. Accordingly, the invention provides a composition comprising PRAME and an oligonucleotide, wherein PRAME has a particle size of 10-40 nm, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nm. In a further embodiment, PRAME has a particle size of 15-25 nm, i.e. 15, 16, 17, 18, 19, 20, 21, 22, 23, 24 or 25 nm. In a further embodiment, PRAME has a particle size of 16-20 nm, i.e. 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 nm.

The invention also provides a composition comprising PRAME and an oligonucleotide, wherein PRAME has a particle size as described above and a polydispersity index between 0.1 and 0.4, i.e. 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39 or 0.40 nm. In a further embodiment, PRAME has a polydispersity index of between 0.2 and 0.3, i.e. 0.20, 0.21, 0.22, 0.23, 0.24, 0.25, 0.26, 0.27, 0.28, 0.29, or 0.30.

Both the hydrodynamic radius and the polydispersity can be measured by dynamic light scattering.

### Dynamic Light Scattering (DLS)

Dynamic light scattering (DLS), which is also known as photon correlation spectroscopy (PCS) or quasi-elastic light scattering (QELS), uses scattered light to measure the rate of diffusion of protein particles in a solution. This motion data is processed to derive a size distribution for the sample, where the size is given by the “Stokes radius” or “hydrodynamic radius” of the protein particle. This hydrodynamic size depends on both mass and shape (conformation). Dynamic scattering allows detection of the presence of very small amounts of aggregated protein (<0.01% by weight).

In dynamic light scattering the time dependence of the light scattered from a very small region of solution, over a time range from tenths of a microsecond to milliseconds is measured. These fluctuations in the intensity of the scattered light are related to the rate of diffusion of molecules in and out of the region being studied (Brownian motion), and the data can be analyzed to directly give the diffusion coefficients of the particles doing the scattering. When multiple species are present, a distribution of diffusion coefficients is seen.

Traditionally, rather than presenting the data in terms of diffusion coefficients, the data are processed to give the “size” of the particles (radius or diameter). The relation between diffusion and particle size is based on theoretical relationships for the Brownian motion of spherical particles, originally derived by Einstein. The “hydrodynamic diameter” or “Stokes radius”, Rh, derived from this method is the size of a spherical particle that would have a diffusion coefficient equal to that of the protein.

Most proteins are not spherical, and their apparent hydrodynamic size depends on their shape (conformation) as well as their molecular mass. Further, their diffusion is also affected by water molecules which are bound or entrapped by the protein. Therefore, the hydrodynamic radius can differ significantly from the true physical size (e.g. that seen by NMR or x-ray crystallography).

Hydrodynamic size and polydispersity index were determined by DLS. In one embodiment, hydrodynamic size and polydispersity index were measured by ZetaNano® from Malvern.

### Pharmaceutically Acceptable Compositions

The invention also provides a method of producing a pharmaceutically acceptable PRAME solution comprising the steps of: (a) carrying out a diluent exchange according to the methods of the invention; and (b) sterilising the formulation produced in step (a).

In one embodiment, the method comprises an additional step (b′) formulating the protein into diluent C prior to step (b). In a further embodiment the method comprises the additional step (c) lyophilising the formulation produced in step (b′) The sterilisation may be via any method known in the art including, but not limited to, UV sterilisation, heat sterilisation or filtration. In one embodiment the sterilisation is achieved using filtration. The filter will usually have a pore size of 0.05-1.0 μm, i.e. 0.05, 0.1, 0.2, 0.3, 0.4, 0.5, 0.6, 0.7, 0.8, 0.9 or 1.0 μm. In a further embodiment, a series of one of more filters may be used to achieve sterilisation and the sterilisation may occur at any point during the steps described above.

Throughout this specification and the claims which follow, unless the context requires otherwise, the word “comprise”, and variations such as “comprises” and “comprising”, will be understood to imply the inclusion of a stated integer or step or group of stated integers or steps but not to the exclusion of any other integer or step or group of integers or steps.

The invention will be further described by reference to the following, non-limiting, figures and examples.

### EXAMPLES

### Example 1

### Characterisation of PRAME

### Isoelectric Point

The isoelectric point (IEP) of the PRAME antigen was determined on purified antigen solubilised in 5 mM Borate buffer pH 9.8-3.15% sucrose by electrophoretic mobility measurement and Zeta potential calculation with ZetaNano® from Malvern. The experimentally obtained value of 6.44 was very close to the value calculated from theoretical amino acid composition (6.41). As the pH of reconstituted vaccine in adjuvant system A (ASA) (Sorbitol) is 8.0, it is expected that the antigen, PRAME, and the CpG present will be globally negatively charged. Therefore, no electrostatic interaction was expected to occur between the two entities.

Material and method for IsoElectric Point (IEP) Determination:

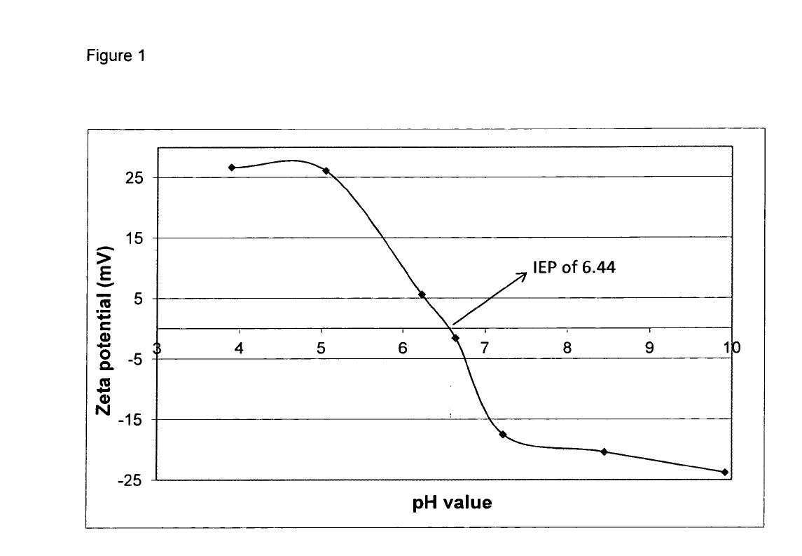

Samples were diluted in 5 mM Borate buffer pH 9.8-3.15% sucrose, and the pH was adjusted to the desired pH with HCl and/or NaOH. The reported zeta potential is the average of 5 consecutive measurements. IEP is the pH at zero zeta potential in the measured “zeta potential versus pH” curve (FIG. 1/21). A reference standard is tested to check performance of the equipment and measurement cells.

The sample measurements were conducted using the experimental conditions shown in Table 1:

TABLE 1

| | | |
|---|---|---|
| Laser wavelength: | 633 | nm |
| Laser power: | 4 | mW |
| Scattered light detected at: | 13° | |
| Temperature: | 22° | C. |

| | |
|---|---|
| Duration: | automatic determination by the soft |
| Number of consecutive | 3-5 consecutive measurements |
| measurements: | |
| pH adjusting solutions if IEP | HCl 0.3M, NaOH 0.5M |
| required | |

### Aggregation State of PRAME

### Aggregation Profile

As part of the proposed plan for stability, the aggregation state of purified PD1/3-PRAME (SEQ ID NO:8)-His bulk is monitored by:
Dynamic Light Scattering (DLS) analysis and
Size-Exclusion High-Performance Liquid Chromatography (SEC) coupled to Multi-Angle Laser Light Scattering (MALLS) detection and to a concentration sensitive detection such as a refractive index (RI).

Purified bulks (PB) of PD1/3-PRAME-His were also characterized by Sedimentation Velocity profiling using Analytical Ultracentrifugation (SV-AUC).

### Size Analysis by DLS

Hydrodynamic size and polydispersity index were determined by DLS for each purified PD1/3-PRAME-His bulk at release (T0). For lots DPRAAPA003, DPRAAPA004 and DPRAAPA005, the values of hydrodynamic size (Z-average, m) and polydispersity were reproducible between the batches. Antigen is aggregated with a size between 16.6 and 19.9 nm; polydispersity ranges from 0.218 to 0.284. No significant change in size can be detected by DLS when the PB lots DPRAAPA003, DPRAAPA004 and DPRAAPA005 are either incubated for 4 hours at 4° C. or stored for 12 months at −70° C. (see m3.2.S.7.3).

### SEC-MALLS Analysis

### Method of Analysis:

SEC analysis using UV, MALLS and RI detectors allows determination of the absolute molar mass (MM) and size (hydrodynamic radius or Rh in nm) of polymers or biopolymers in solution without reference to calibration standards and without prior assumptions about their molecular conformation. It is also a sensitive method for detecting aggregates even in a small amount.

### Results and Discussion:

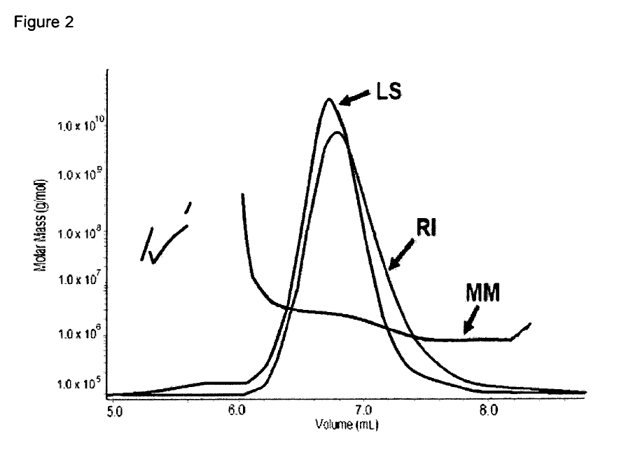

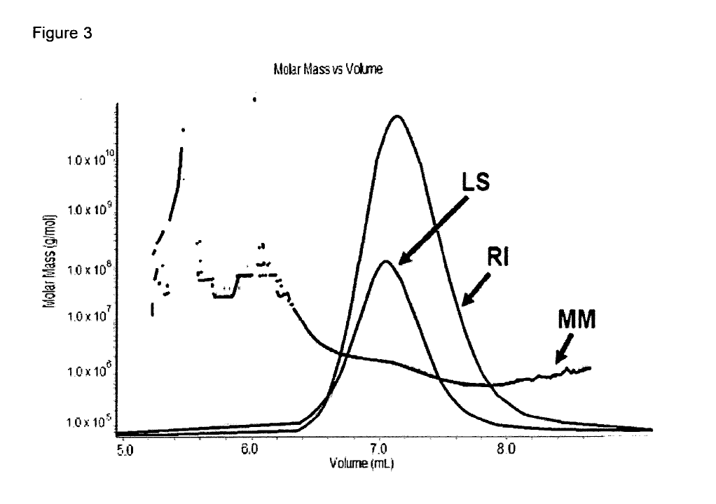

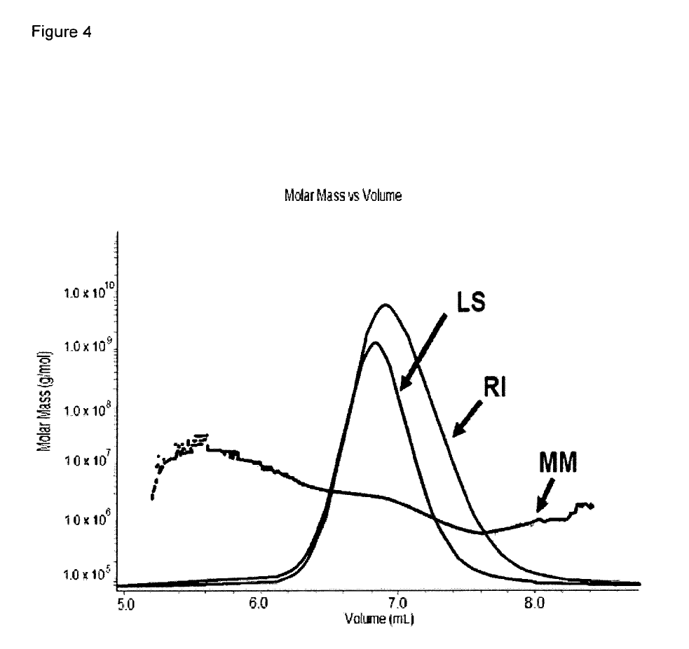

This analysis was performed on the PB lots DPRAAPA003, DPRAAPA004 and DPRAAPA005. FIG. 2/21, FIG. 3/21 and FIG. 4/21 show the light scattering (LS) profiles, RI profiles, and molar mass (MM) distribution in function of the elution volume obtained by SEC-MALLS analyses of the three GMP lots at release.

In the final buffer (5 mM borate, 3.15% sucrose, pH 9.8), PBs consist of polydisperse, soluble aggregates eluting between 6.0 and 7.7 mL and by MM values varying between 600 and 3,000 kDa.

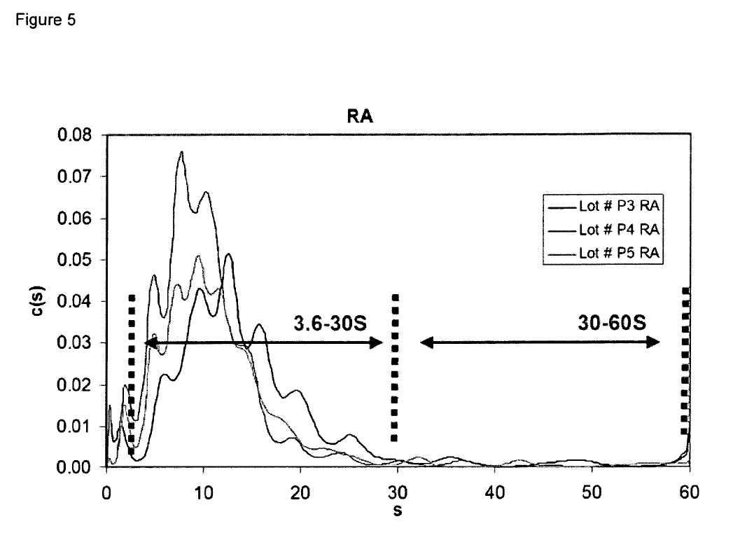

Aggregates with higher molecular mass elute between 5.0 and 6.0 mL, but they represent a small fraction of the total purified protein bulk. This was confirmed by SV-AUC analysis shown below in FIG. 5/21 and Table 2.

### SV-AUC Analysis

### Method of Analysis:

To ensure that the aggregation profile of a biopolymer in solution is not influenced and/or caused by putative interactions between the solution to be analyzed and the chromatographic bed, the protein aggregation status and distribution was analyzed directly in solution and in real time by analytical ultracentrifugation. Briefly, the reference (protein buffer) and sample solutions are centrifuged at high speed (35,000 rpm) and their absorbance at 280 nm recorded. The acquired data reflect the spatial concentration gradients of sedimenting species and their evolution with time generated after applying the centrifugal field. Sedimentation depends both on the size and shape of the protein. Time course analysis of the sedimentation process also termed sedimentation velocity (SV-AUC) allows the calculation of the sedimentation coefficients (s). The s values are reported in Svedberg (S) units, one unit corresponding to 10-13 seconds.

For purified PD1/3-PRAME-His bulk, the sedimentation coefficient distribution c(s) were obtained using Sedfit software.

### Results and Discussion

FIG. 5/21 shows the results of the SV-AUC analysis performed on PB lots DPRAAPA003, DPRAAPA004 and DPRAAPA005 at release.

Table 4 shows the correspondence between each aggregate detected in FIG. 5/21 and its respective sedimentation coefficient and molecular weight. This qualitative interpretation is based on the fact that the 72-kDa monomeric PD1/3-PRAME-His protein forms globular compact aggregates as demonstrated by electron microscopy. For such globular aggregates, a classical frictional ratio f/f° of 1.2 can be attributed.

TABLE 2
Correspondence between the aggregates and their
sedimentation coefficient and molecular weight.

| Aggregate Number (N) | 1 | 2 | 3 | 4 | 5 | 6 | 8 | 10 | 12 | 20 | 40 |
|---|---|---|---|---|---|---|---|---|---|---|---|
| Sedimentation | 4.2 | 6.8 | 8.8 | 11 | 12.5 | 14 | 17 | 20 | 22 | 31 | 50 |
| coefficient (S) | | | | | | | | | | | |
| MW (kDa) | 72 | 144 | 216 | 288 | 360 | 432 | 576 | 720 | 864 | 1440 | 2880 |

As shown in FIG. 5/21 and in Table 2, the majority of purified protein bulk is represented in solution by a polydisperse population (characterized by sedimentation coefficients between 3.6 and 30 S) ranging from the 72-kDa monomeric form to aggregated complexes consisting of 20 monomeric molecules (MW=1,440 kDa). The mean sedimentation coefficient (s bar) obtained at release for PB from lots DPRAAPA003, DPRAAPA004 and DPRAAPA005 were 13.5, 10.2 and 11.1 S, respectively.

The 3.6-30-S polydisperse population accounts for 95%, 97% and 96% of the total PB from lots DPRAAPA003, DPRAAPA004 and DPRAAPA005, respectively. The remainder is represented by higher aggregates characterized by higher sedimentation constants (30 to 60 S).

In conclusion, the three GMP lots of PD1/3-PRAME-His have similar sedimentation coefficient distributions.

### Example 2

### Evidence of Interaction with CpG

### SDS-Page/WB Anti-PRAME (Additional Band 7 kDa Above Monomer)

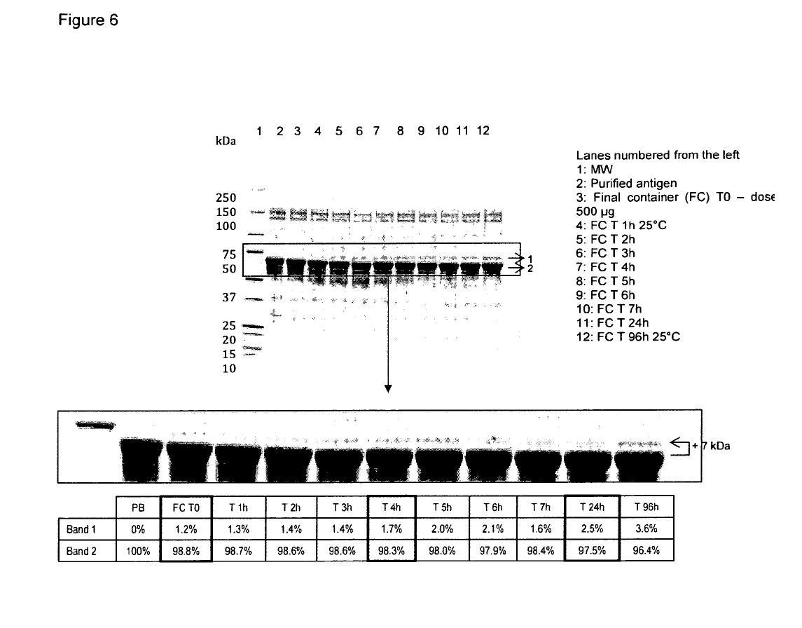

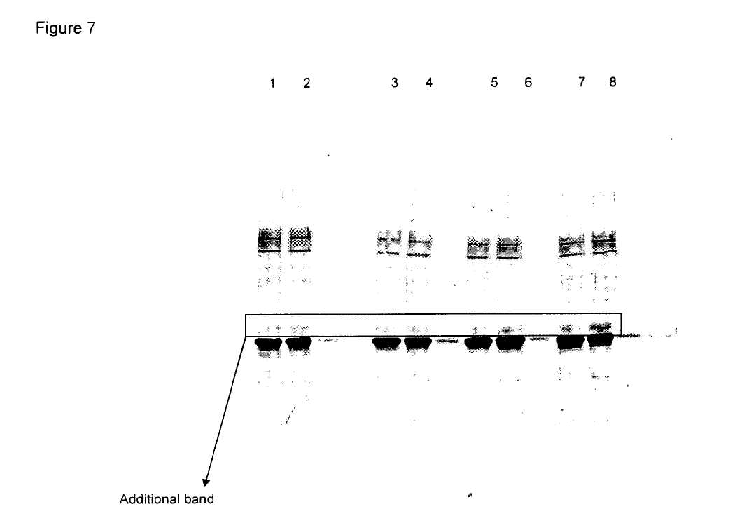

SDS-PAGE analysis was conducted on Final Container reconstituted in ASA (Sorbitol). As illustrated in FIG. 6/21, additional band (band 1) is detected in Final Container at T0 (cf. lane 3). Based on analysis by densitometry (Biorad GS-700 Imaging Densitometer™), this additional band is characterized by a MW of 7 kDa higher than PRAME monomer band (band 2) and its intensity increases over time but remains below 4% (w/w versus monomer) 96 h post-reconstitution. Western Blot analysis using specific anti-PRAME antibody (FIG. 7/21) confirmed that the additional band is product-related and that its intensity slightly increases over time (lane 3 vs. 7).

### Isothermal Titration Calorimetry

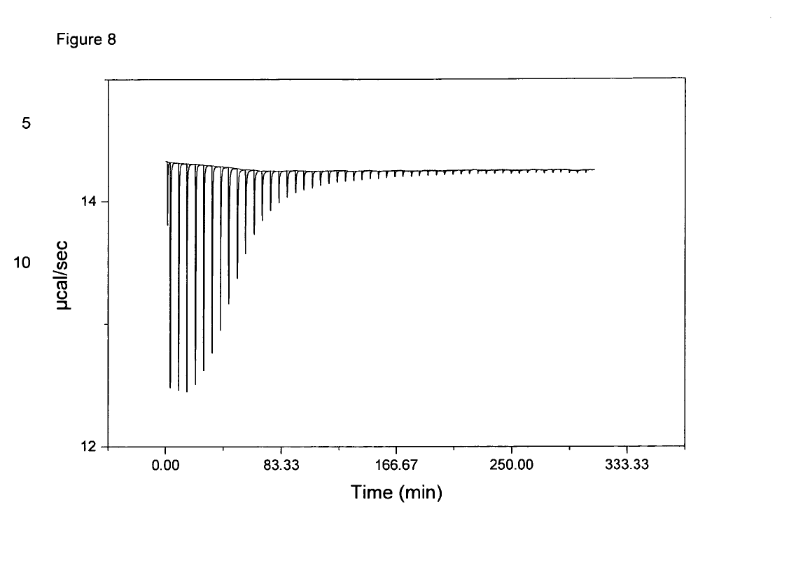

ITC measures directly the energy (heat) associated with a chemical reaction triggered by the mixing of two components. A typical ITC experiment is carried out by the stepwise injection of a solution containing one reactant into the reaction cell containing the other reactant. The ITC setup used for the study of the PRAME antigen/CpG complex implied the injection of CpG liquid bulk (diluted in the reconstituted vaccine buffer (borate 5 mM sucrose 3.15% pH 9.8)) into a solution of PRAME antigen (in the same buffer). A typical titration profile is presented in FIG. 8/21

As observed in FIG. 8/21, each injection of CpG into the PRAME solution results in a negative peak indicating a very significant exothermic binding reaction. Because the amount of uncomplexed protein available progressively decreases after each successive injection, the magnitude of the peaks becomes smaller until complete saturation is reached. Of note, control experiments (data not shown) consisting in the injection of PRAME buffer without the antigen gives a flat profile.

The amount of CpG needed to reach the plateau of saturation is equivalent to a mass ratio CpG/antigen ranging between 0.05 and 0.10 in good agreement with the complex stoichiometry determined by ultracentrifugation.

### Materials and Methods

The isothermal titration calorimeter is composed of two identical cells made of a highly efficient thermal conducting material. Temperature differences are monitored between a reference cell (filled with water) and a sample cell (containing the oil-in-water emulsion, AS03). Measurements consisted of time-dependent input of power (expressed as μcal/s) required to maintain equal temperatures between the reference and sample cells. Set up and general protocol used for the ITC instrument follow specifications provided by the manufacturer (MicroCal, USA). All samples prior to use were degassed for 5 minutes to minimise data interference due to the presence of bubbles. CpG was filled into the injection syringe and titrated into the sample cell containing the antigen. Titration comprised of 1 injection of 2 μl followed by 24 successive injections of 10 μl, with a 6 minute delay between each injection. The antigen was loaded into the sample cell up to the fill level (the sample cell in this instrument has an internal volume at the fill level of 1404 μl).

Control titrations for CpG and antigen alone are always included in the testing protocols.

### Rate-Zonal Ultracentrifugation

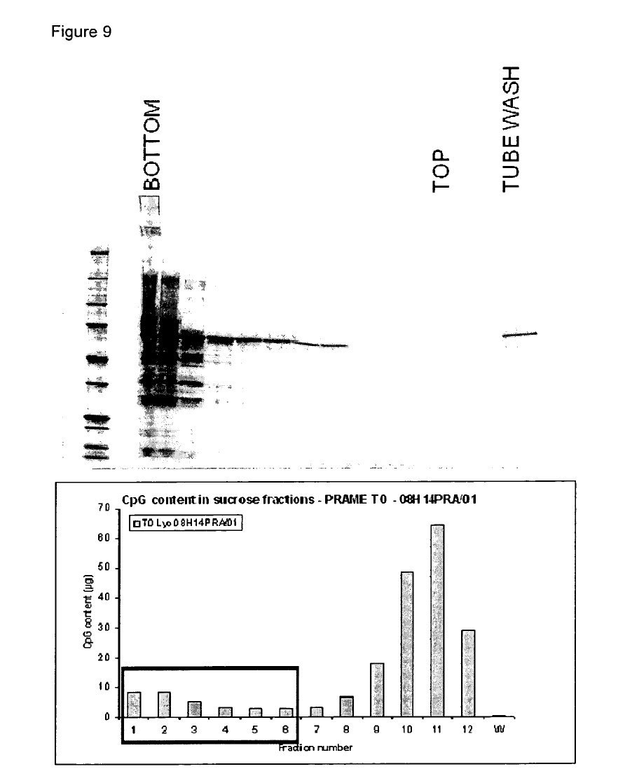

In the aim of isolating a PRAME/CpG complex from free antigen and CpG, rate zonal configuration was performed. Samples were loaded on top of a linear sucrose gradient and separated based on their sedimentation rate. Unlike ITC as described above, this setup additionally allows the analysis of reconstituted vaccine samples. After optimization of the experimental conditions, distribution of antigen and CpG in a sucrose gradient was observed as shown in FIG. 9/21.

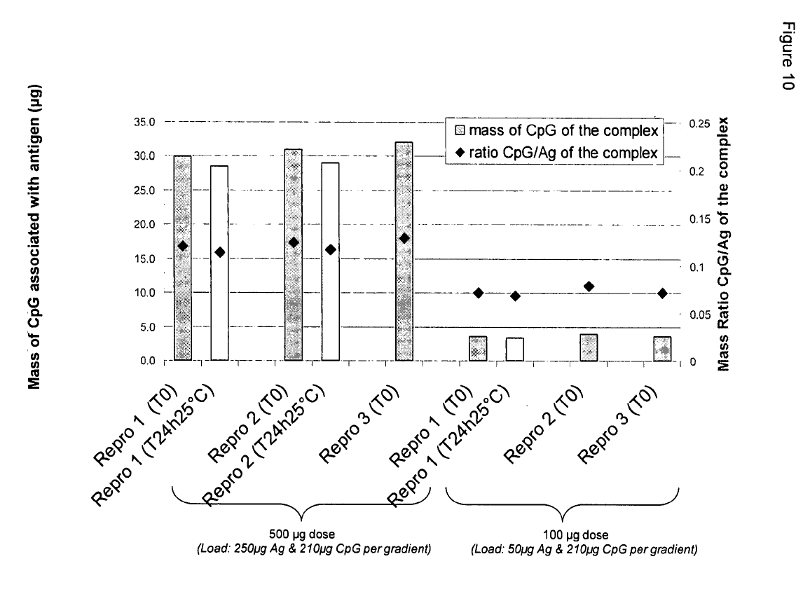

Subsequently, SDS-PAGE was replaced by RP-HPLC-UV to obtain a quantitative determination of the antigen in the sucrose fractions. To determine whether the CpG/antigen interaction is subject to significant batch-to-batch variations, three repro lots containing 500 μg of PRAME per dose and 3 lots of 100 μg/dose were submitted to rate zonal ultracentrifugation and further analyzed to determine the stoichiometry of the CpG/antigen complex. These are shown in FIG. 10/21.

The results show that the amount of CpG associated with the antigen is very similar between lots. As expected, decreasing the antigen dose from 500 to 100 μg leads to a decrease of the amount of CpG in the complex. The stoichiometry, as expressed by the CpG/Ag mass ratio is however not directly proportional to the antigen dosage. The pre-incubation of the sample at 25° C. for 24 h doesn't influence the complex stoichiometry.

These results strongly suggest that CpG consistently interacts with PRAME.

### Materials and Methods

In to a 14×89 mm centrifuge tube, 5 ml of a 25% sucrose solution was added under the same volume 5 ml of a 5% sucrose solution. The tube was loaded on the Master Gradient to proceed a continuous gradient 5-25%. A volume of 1 ml was removed on the tube of the gradient. This volume was replaced at the bottom of the gradient by 1 ml of 50% sucrose solutions. Samples (or controls) were pre-incubated for 15 min at 25° C. to allow all samples, including those stored at 4° C., to begin the centrifugation at equivalent temperatures. 250 μl ml aliquot of the sample was loaded on the top of the gradient. The gradient was centrifuged at 100 000 relative centrifugal force (rcf) for 67 h at 4° C.

### Sucrose Fraction Collection

Fractions resulting from ultracentrifugation were collected by pipetting from the top of the tube. Successive suction of 1-mL fractions was performed. Upon collection, the fractions were stored at 4° C. until subsequent analysis

### Fraction Analysis

### Detection of ASCI Antigens

Antigens were analyzed by SDS-PAGE. Alternatively, RP-HPLC-UV was employed for quantitative purpose.

### Detection of Liposome Components.

Liposomes localization was performed by determination of cholesterol (colorimetric kit, Roche Diagnostics). Alternatively, IP-HPLC-UV was used.

### Detection of CpG

CpG was determined by IEX-HPLC-UV.

### Samples

### Antigen PBs: Prame: R03.

Final containers (lyophilized cakes): Prame: 08H14PRA01, 08H20PRA01, 08I09PRA01 (500 μg/HD)

08H14PRA02, 08H20PRA02, 08I09PRA02 (100 μg/HD). AS01B:DA1BA008A

CpG liquid bulk: DCPGAFA003

### ELISA (Interference)—PD-PRAME-his:Antigen Content by ELISA on pH I/II Material

In order to further investigate the observed interaction between PRAME and CpG a sandwich ELISA based on the use of a mAb anti-PRAME and a polyclonal antibody (Pab) anti protein D (PD) was developed to measure the antigen content.

Using this ELISA to test the antigen content of the PB gives the expected value of PBs (PB). However, when the ELISA is applied to Final Container (FC), a loss of antigenicity is seen.

N.B. The PB contains the antigen in a Borate 5 mM sucrose 3.15% buffer while the FC contains CpG (420 μg/dose), poloxamer 188 at 0.24%, sucrose 4% and Tris 16 mM

To test whether this observed effect was related to the CpG, the PB was spiked with increasing doses of CpG and the antigen content was measured by ELISA, as shown in table 3.

TABLE 3

| | | expect- | | recov- |
|---|---|---|---|---|
| | | ed conc. | result | ery % |
| | | (Lowry) | ELISA | (ELISA/ |
| | non linearisé | μg/ml | (μg/ml) | Lowry) |
| A | std R01 1604 μg/ml | 1604 | 1594 | 99 |
| B | R02 1851 μg/ml + CpG 1000 μg/ml | 1851 | 67 | 4 |
| C | R02 1851 μg/ml + CpG 100 μg/ml | 1851 | 489 | 26 |
| D | R02 1851 μg/ml + CpG 10 μg/ml | 1851 | 1427 | 77 |
| E | R02 1851 μg/ml + CpG 1 μg/ml | 1851 | 1697 | 92 |
| F | R02 1851 μg/ml + CpG 0.1 μg/ml | 1851 | 1448 | 78 |
| G | R02 1851 μg/ml + CpG 0.01 μg/ml | 1851 | 1542 | 83 |
| H | R02 1851 μg/ml | 1851 | 1485 | 80 |

These results show that the addition of CpG in the PB is associated with decreases the antigenicity especially from the concentration comprised between 10 and 100 μg/ml suggesting a change of conformation of the antigen when CpG is added.

### Material and Method

This method is based on a “Sandwich” ELISA: Before addition of the antigen PDPRAME-his (repro lot R02) the immunoplate is coated with a mouse monoclonal antibody directed against PRAME (MK1H8C8 diluted 500×) overnight at 4° c. After reaction with the antigen for 90′ at 37° C., a rabbit polyclonal antibody directed against PD (LAS98733) is added for 90′ at 37° C. After reaction with the Pab for 90′ at 37° C., a biotinylated donkey whole antibody against rabbit immunoglobulins is added for 90′ at 37° c. The antigen-antibody complex is revealed by incubation with a streptavidin-biotinylated peroxidase complex for 30′ at 37° c. This complex is then revealed by the addition of tetramethyl benzidine (TMB) for 15′ at Room Temperature and the reaction is stopped with 0.2 M H2SO4. Optical densities are recorded at 450 nm.

The concentrations of samples are calculated by SoftMaxPro™ referring to a standard antigen (repro lot R01 at 1604 μg/ml)

### SEC-HPLC (Interference)

### Material and Methods

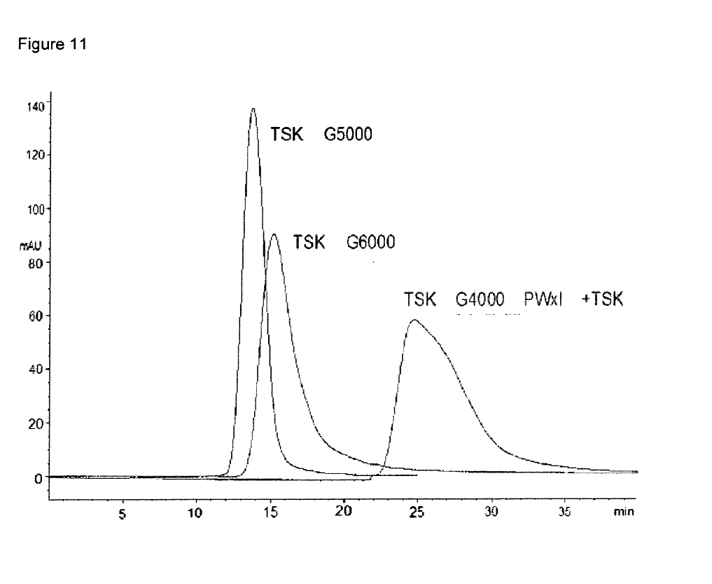

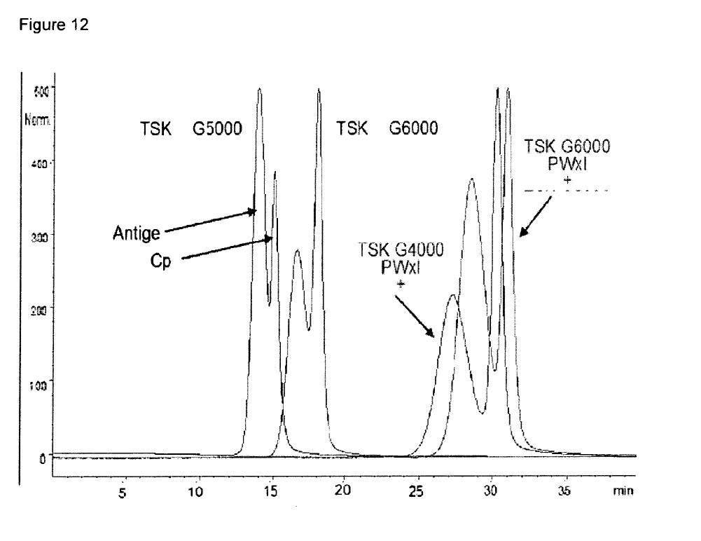

Size-exclusion chromatography (SEC) also called gel permeation or gel filtration chromatography is a method separating molecules in solution based on their size or shape. Antigen size follow-up through formulation process is one of the success criteria when developing a vaccine candidate. The first objective was therefore to develop an analytical SEC method for this purpose. As CpG is added to the vaccine candidate, purified antigen alone (FIG. 11/21) or spiked with CpG solution (FIG. 12/21) was injected on several SEC columns (alone or in combination) from Tosoh supplier equilibrated in 5 mM Borate buffer pH 9.8-3.15% sucrose (=buffer of the purified antigen) at a flow-rate of 0.5 ml/min and with UV detection at 220 nm. Guard column recommended by Tosoh supplier was also used with each column or combination of columns. As illustrated in FIG. 12/21, injection of purified antigen spiked with CpG solution on a single column (TSK G5000 PWxI or TSK G6000PwxI) led to significant overlapping of molecules peaks while a combination in series of two columns improves the resolution. Combination of two columns, TSK G4000PWxI+G 6000 PWxI was therefore selected as SEC analytical tool for the follow-up of formulation development.

### Characteristics of the Columns Tested

TSKgel PWxI™ guard column: 6 mm internal diameter (ID)×4 cm length (L)-Tosoh-Ref 08033
TSK G4000 PWxI™ column: 7.8 mm ID×30 cm L-Tosoh—Ref 08022
TSK G6000 PWxI™ column: 7.8 mm ID×30 cm L-Tosoh—Ref 08024
TSK G5000 PWxI™ column: 7.8 mm ID×30 cm L-Tosoh—Ref 08023

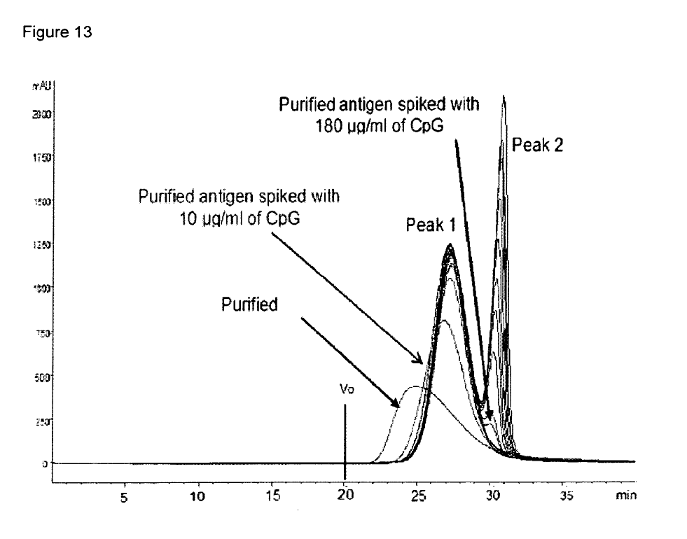

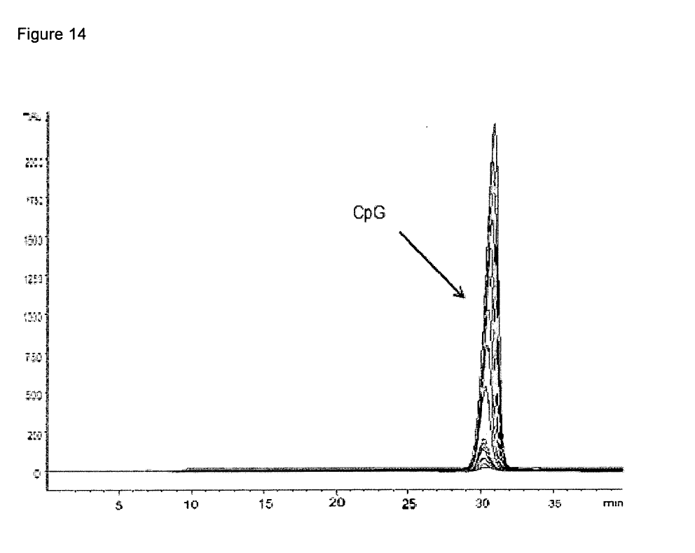

As illustrated in FIG. 13/21, increase of retention time and surface area for purified antigen (1.68 mg of protein/ml) is already observed after spiking with CpG solution up to 10 μg/ml of. The higher retention time would suggest a lower size (=indirect indication of the positive effect of CpG on antigen solubility). Purified antigen was therefore further spiked with increasing concentrations of oligonucleotide solution (from 10 up to 1050 μg/ml). Surface area of first elution peak (peak 1) increases for CpG concentrations up to 60 μg/ml, then remains constant for all upper spiking concentrations up to 1050 μg CpG/ml. A second peak (peak 2) corresponding to free CpG (FIG. 14/21 representing UV profiles obtained after injection of different concentrations of CpG solution) is detected from 180 μg/ml of CpG.

### Example 3

### Excipient Screening

Starting from purified antigen solubilised in 5 mM Borate pH 9.8-sucrose 3.15%, 25 excipients were assessed for the stabilization of the antigen size upon storage at +4° C. and at +22° C. The listing of the excipients and the concentrations tested are listed in the table 4

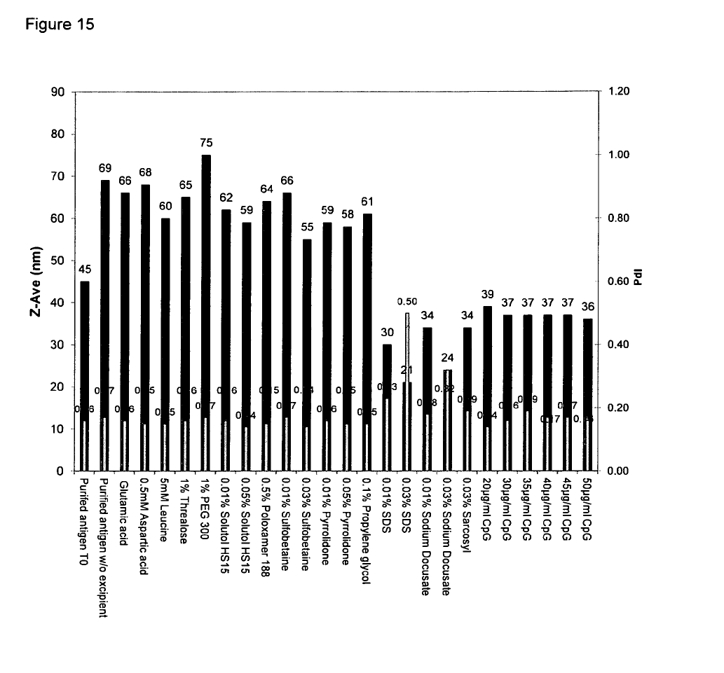

First selection of the candidates was performed through visual observation and size analysis by dynamic light scattering after 24 h storage at 22° C. (cf. table 4 and FIG. 15/21). Amongst all the excipients tested, only four of them allowed antigen size stabilization: SDS 0.01%, Sodium Docusate 0.01%, Sarcosyl 0.03% and CpG (from 20 μg/ml to 50 μg/ml)

TABLE 4
Table 4: Listing and concentrations of the excipients tested for the
stabilization of the antigen size and visual observation on purified
antigen samples spiked or not with excipient after 24 h storage at 22°
C. PB = Purified antigen in 5 mM Borate pH 9.8 - sucrose 3.15%.

| | | | Visual |
|---|---|---|---|
| | | | observation |
| Excipient | Concentration | unit | 24 h 22° C. |
| L-glycine | 10 | mM | cloudy |
| | 100 | mM | opaque |
| L-Histidine | 10 | mM | cloudy |
| | 100 | mM | opaque |
| L-Glutamic acid | 1 | mM | slightly cloudy |
| | 10 | mM | cloudy |
| L-Aspartic acid | 0.5 | mM | slightly cloudy |
| | 100 | mM | opaque |
| L-Leucine•2HCl | 5 | mM | slightly cloudy |
| | 30 | mM | cloudy |
| MgCl2•6H2O | 1 | mM | opaque |
| | 50 | mM | opaque |
| MgSO4•7H2O | 1 | mM | opaque |
| | 50 | mM | opaque |
| Trehalose•2H2O | 1 | % | slightly cloudy |
| | 5 | % | cloudy |
| Polyethyleneglycol 300 | 1 | % | slightly cloudy |
| | 5 | % | cloudy |
| Polyethyleneglycol 6000 | 1 | % | cloudy |
| | 5 | % | cloudy |
| (Myo-)Inositol | 1 | % | cloudy |
| | 5 | % | very cloudy |
| D-Mannitol | 1 | % | opaque |
| | 5 | % | opaque |
| Sorbitol | 1 | % | opaque |
| | 5 | % | very cloudy |
| SDS Sodium Lauryl Sulfate | 0.01 | % | clear |
| | 0.03 | % | clear |
| Sodium docusate | 0.01 | % | clear |
| | 0.03 | % | clear |
| Solutol HS15 | 0.01 | % | very slightly cloudy |
| | 0.05 | % | very slightly cloudy |
| Triton X-100 (Octoxynol 9) | 0.3 | % | slightly cloudy |
| | 0.5 | % | slightly cloudy |
| Poloxamer 188 (Lutrol F68) | 0.05 | % | slightly cloudy |
| | 0.5 | % | slightly cloudy |
| Sulfobétaïne (SB3-12) | 0.01 | % | slightly cloudy |
| | 0.03 | % | slightly cloudy |
| 2-Pyrrolidone | 0.01 | % | slightly cloudy |
| | 0.05 | % | slightly cloudy |
| Propyleneglycol | 0.1 | % | slightly cloudy |
| | 1.5 | % | slightly cloudy |
| a-tocopheryl | 1 | mM | clear |
| Hydrosuccinate (Vit E | 5 | mM | clear |
| succinate) | | | |
| CPG | 0 | μg/ml | slightly cloudy |
| | 20 | μg/ml | slightly cloudy |
| | 30 | μg/ml | slightly cloudy |
| | 35 | μg/ml | slightly cloudy |
| | 40 | μg/ml | slightly cloudy |
| | 45 | μg/ml | slightly cloudy |
| | 50 | μg/ml | slightly cloudy |
| Sarcosyl | 0.03 | % | slightly cloudy |
| Purified antigen (=PB) | 1500 | μg/ml | slightly cloudy |
| | 1250 | μg/ml | slightly cloudy |
| | 1125 | μg/ml | slightly cloudy |

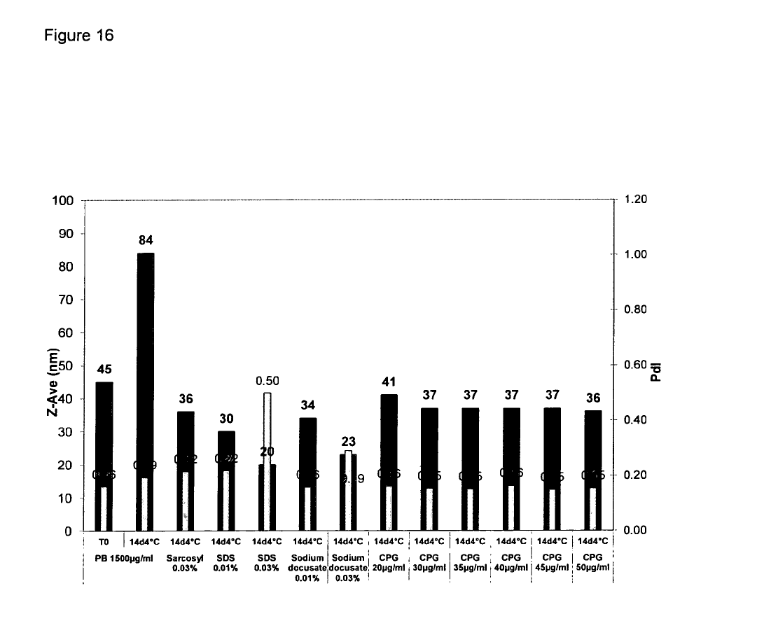

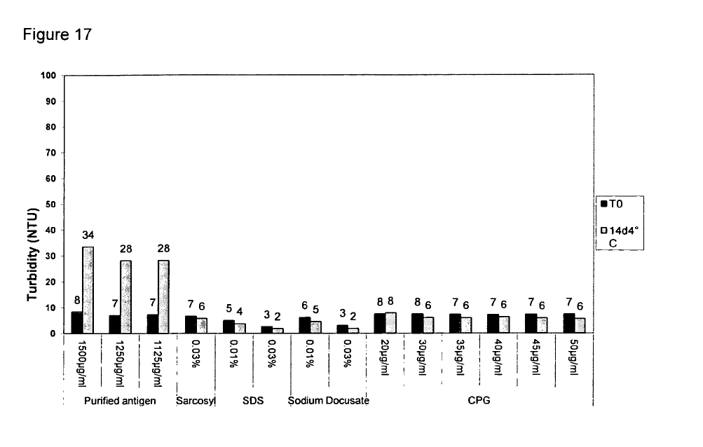

Additional stability data were generated on the four selected candidates (SDS, Sodium Docusate, Sarcosyl and CpG) up to 14 days at 4° C. As illustrated by the FIG. 16/21, antigen size remains stable in presence of the four excipients while it increases up to 84 nm for the non-spiked purified antigen (═PB sample). Turbidity measurement on the same samples (cf. FIG. 17/21) confirmed an evolution only for the non-spiked purified antigen.

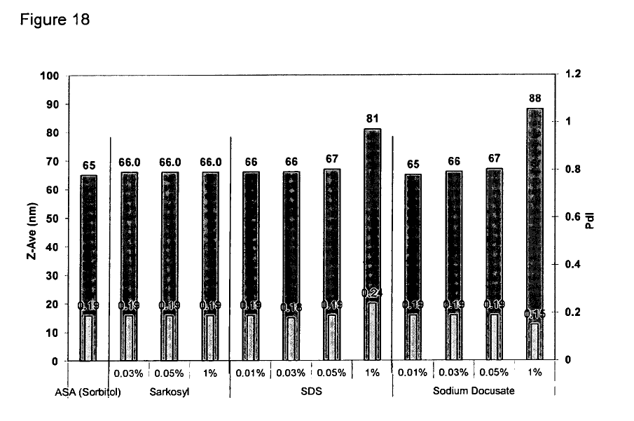

Next step included the evaluation of ASA (Sorbitol) compatibility (Liposome size and QS21 quenching) with the ionic detergents (Sarsosyl, SDS and Sodium Docusate). Liposome size increases in presence of 1% SDS or Sodium Docusate and remains stable up to 1% of Sarcosyl (cf. FIG. 18/21). The three ionic detergents alone induce lysis of red blood cells.

### Conclusions:

Taking into account that SDS, Sodium Docusate and Sarcosyl alone induced red blood cells lysis and the limited or non injectability of these excipients, the use of CpG as an excipient in the PB was prioritized.

### Example 4

### Effect of CpG on PRAME PB Solubility

### Introduction

This example summarizes the data collected to document the solubilizing effect of the CpG7909 on PRAME antigen in final purification buffer.

When put in the final buffer (5 mM Borate-3.15% Sucrose buffer) it was observed that PRAME antigen was very likely to form insoluble aggregates and cause precipitation. An effort was made to find an excipient to enhance solubility of the protein. Among a panel of excipient candidates, CpG was evaluated and demonstrated an (unexpected) improvement of PRAME solubility in the final buffer.

The results of CpG concentration screening experiment are described below.

### First CpG Concentration Screening—Dialysis Trials

### Experimental Design

The aim is to start with a sample of PB of PRAME in a Borate-Sucrose buffer containing 300 ppm Lauryl-Sarcosyl (LS). This amount of detergent has demonstrated its ability to keep the protein soluble. Then we use a dialysis operation to eliminate the LS and replace it with an increasing amount of CpG. After the buffer exchange, the aggregation evolution of the product is monitored by DLS to estimate the amount of CpG needed to maintain a steady aggregation state.

### Material & Buffers

Starting Material: PB PRAME in buffer 5 mM Borate/3.15% sucrose/300 ppm Lauryl-Sarcosyl-pH 9.8 (designated R23/1). Four samples of 2 ml of PB were spiked up to tested concentration using a concentrated solution of CpG: 0 μg/ml CpG (control); 50 μg/ml CpG; 200 μg/ml CpG; 400 μg/ml CpG. Dialysis buffer=5 mM Borate/3.15% Sucrose-pH 9.8 (2×1 L per assay). Dialysis cassette [Pierce Slide-A-Lyzer 20,000 MWCO]

### Method

2 ml of sample were introduced in a dialysis cassette. Each cassette was submerged in a recipient containing 1 L of dialysis buffer. The dispositive was put under gentle agitation (magnetic stirrer) at room temperature.

The first 1 L of dialysis bath was replaced by 1 L of new buffer after 2 hours and left under gentle agitation overnight at room temperature.

The following day, the sample in the cassette was recovered in an eppendorf container (PP) and stored at +4° C. for further analysis.

### Analytics

Analysis was performed by: visual aspect; CpG content by HPLC-IEX-UV (Dionex DNAPac PA200™ column) to monitor the remaining CpG content; Lauryl-Sarcosyl content by RP-HPLC-UV (Waters SunFire C18 column) to insure that LS (initial solubilisating detergent) is well removed; Dynamic Light Scattering (DLS) (ZetaNano® from Malvern) is measured on dialysed product after a 24 h and a 72 h storage period at +4° C. to follow-up size evolution.

### Results

Visual aspect: all samples are limpid after dialysis operation

TABLE 5
Size by DLS after 24 h and 72 h/+4° C.

| | After 24 h/RT dialyse | | After 72 h/+4° C. | |
|---|---|---|---|---|
| | Z- | Polydispersity | Z- | |
| Sample | ave(nm) | index (PdI) | ave(nm) | PdI |
| Negative Ctrl | 22.8 | 0.17 | — | — |
| (no dialyse) | | | | |
| +0 μg CpG | 56.6 | 0.134 | — | — |
| (Positive ctrl) | | | | |
| +50 μg CpG | 24.9 | 0.18 | 27.6 | 0.17 |
| +200 μg CpG | 20.4 | 0.21 | 20.6 | 0.26 |
| +400 μg CpG | 20.4 | 0.21 | 21.04 | 0.24 |

TABLE 6
Lauryl-Sarcosyl content by RP-
HPLC + CpG content by IEX-HPLC

| | LS | CpG | |
|---|---|---|---|
| | content | content | CpG |
| Sample | ppm | (μg/ml) | Recovery % |
| PRAME Control | 293 | — | — |
| (T0 − no dialysis) | | | |
| PRAME + 50 μg CpG | <0.5 | 39 | 78 |
| (+dialysis) | | | |
| PRAME + 200 μg CpG | <0.5 | 153 | 77 |
| (+dialysis) | | | |
| PRAME + 400 μg CpG | <0.5 | 317 | 79 |
| (+dialysis) | | | |
| BO3-Sucrose + 100 μg CpG | — | 94.2 | 94 |
| (no dialysis) | | | |
| BO3-Sucrose + 100 μg CpG | — | 716 | 72 |
| (+dialysis) | | | |

The LS is well eliminated during dialysis operation (measure below LOQ for dialysed sample) and the CpG stays on the inner side of the dialysis cassette with an average recovery around 80% even in absence of PRAME.

We observed that a quantity of CpG between 50 and 200 μg/ml maintains the particle size at approximately 20 nm after removal of Lauryl-Sarcosyl. This observation suggests that the PRAME-CpG interaction is beneficial to the solubility of PRAME.

### Second CpG Concentration Screening—UltraFiltration Trials

### Objective

The screening of the CpG quantity required to keep PRAME soluble using an UltraFiltration system for assessment.

### Material & Buffers

Starting Material: HydroxyApatite Flow-through (HA-FT) Antigen fraction from R25/2 purification.

Sample Buffer composition=20 mM Tris-6M Urea-0.5% Lauryl Sarcosyl-50 mM PO4-˜80 mM Imidazole

4 samples of approximately 70 ml of HA-FT are processed in 4 independent UF experiments. A small volume of aqueous concentrated solution of CpG is added to reach following concentration: 50 μg/ml CpG→UF-A (no CpG in Diafiltration buffer); 75 μg/ml CpG→UF-B (no CpG in Diafiltration buffer); 100 μg/ml CpG→UF-C (no CpG in Diafiltration buffer); 50 μg/ml CpG→UF-D (50 μg/ml CpG in Diafiltration buffer)

The CpG spiked samples are incubated 1 h at Room Temperature under very mild agitation prior Ultrafiltration

Diafiltration buffers: 5 mM Borate/3.15% Sucrose-pH 9.8 (for UF-A/B/C); 5 mM Borate/3.15% Sucrose+50 μg/ml CpG-pH 9.8 (for UF-D)

UltraFiltration cassette [Minimate™-Omega-from Pall Cut-off 30 kD]-surface 50 cm²

UltraFiltration system [KrossFlo™ from JM JM Bioconenct II]: including a peristaltic pump, appropriate tubing to accommodate the UF cassette and 3 pressure gauges

### Method

70 ml of HA-FT sample spiked to appropriate CpG content are diafiltered against 15 Diafiltration Volume (Vol total Diaf buffer=1050 ml) of diafiltration buffer.

UF-conditions:

Recirculation flow-rate=35 ml/min

TMP regulation: 8 psi for DV 1->8 then 12 psi for DV 9->15 by adjusting retentate counter-pressure valve

No concentration of Ag is done—only diafiltration

At end of operation: final retentate product is kept at +4° C. for further analysis

A clean in place (CIP) of 2×30 min under NaOH 0.5N (static) is done between different UF

### Analytics

Analysis was performed by: CpG content by HPLC-IEX-UV (Dionex DNAPac PA200 column); Lauryl-Sarcosyl content by RP-HPLC-UV (Waters SunFire C18 column); Dynamic Light Scattering (DLS) (ZetaNano® from Malvern) is measured directly after UF and after 1 week stored at +4° C. and Room Temp to follow-up size evolution.

### Results

TABLE 7
DLS measures for each of the concentrations tested -
“$” represent s the sample which comes from
the UF run with the corresponding letter as described above.

| | | Z-average | |
|---|---|---|---|
| Buffer Conditions | Stability Point | (nm) | PdI |
| $A + 50 μg/ml CpG | To | 22.47 | 0.102 |
| $A + 50 μg/ml CpG | 1 w/RT | 23.85 | 0.095 |
| $A + 50 μg/ml CpG | 1 w/+4° C. | 24.19 | 0.106 |
| $B + 75 μg/ml CpG | To | 18.11 | 0.121 |
| $B + 75 μg/ml CpG | 1 w/RT | 18.98 | 0.104 |
| $B + 75 μg/ml CpG | 1 w/+4° C. | 26.55 | 0.289 |
| $C + 100 μg/ml CpG | To | 16.62 | 0.135 |
| $C + 100 μg/ml CpG | 1 w/RT | 16.29 | 0.108 |
| $C + 100 μg/ml CpG | 1 w/+4° C. | 16.02 | 0.134 |
| $D + Tp Diaf: 50 μg/ml CpG | To | 10.61 | 0.247 |
| $D + Tp Diaf: 50 μg/ml CpG | 1 w/RT | 11.28 | 0.221 |
| $D + Tp Diaf: 50 μg/ml CpG | 1 w/+4° C. | 10.84 | 0.289 |

TABLE 8
LS content & CpG content

| | LS | CpG | |
|---|---|---|---|
| | content | content | |
| Sample | ppm | (μg/ml) | Recovery % |
| Internal Control | 161 | — | — |
| (150 ppm buffer) | | | |
| $A + 50 μg CpG | <0.5 | 40 | 80 |
| $B + 75 μg CpG | — | 49 | 65 |
| $C + 100 μg CpG | — | 79 | 79 |

LS is fully removed after 15 diafiltration volume (even in presence of CpG). CpG recoveries measured between 65-80%

### Conclusions

The solubilizing effect of CpG was observed when using an Ulrafiltration.

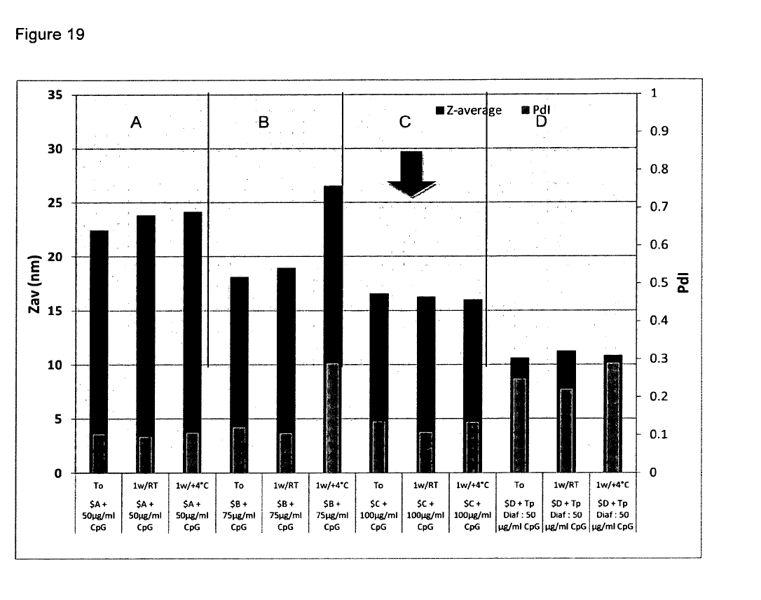

The green arrow in FIG. 19/21 represents a spiking concentration of 100 μg/ml CpG in HA-FT before UF-R is selected as the appropriate concentration because the samples are the most stable over time, i.e. there is no increases in size after 1 week.

It does not appear to be necessary to add CpG in diafiltration buffer (UF-D) as the majority of CpG remains on sample side and is sufficient to keep product soluble. Spiking of HA-FT with 100 μg CpG on a 1 L scale purification was then investigated.

### Verification of 100 μg CpG Spiking Option at 1 L Scale

### Objective

Verify the feasibility of a spiking with 100 μg/ml CpG with a 1 L scale process (final development scale)

### Procedure

Run R26/1: full PRAME purification at 1 L scale+spiking with 100 μg/ml CpG in HA-FT prior UF
Check stability of final product with classical stress tests
Stability 1 week @-70° C./+4° C./RT and 37° C.
2 to 3 Freeze/Thaw cycles (−70° C.-RT)
check LS removal by RP-HPLC-UV
check CpG content (IEX-HPLC-UV) at end of UF in 1 L scale condition

### Results

TABLE 9
Size evolution follow-up by DLS measures

| Stress Conditions | Zav (nm) | PdI |
|---|---|---|
| T0 | 18.7 | 0.14 |
| 1 w/−70° C. | 19.6 | 0.15 |
| 1 w/+4° C. | 21 | 0.24 |
| 1 w/RT | 20.9 | 0.2 |
| 1 w/37° C. | 19.2 | 0.1 |
| 2 cycles F/T | 19.6 | 0.17 |
| 3 cycles F/T | 40.9 | 0.29 |

There was no significant increase in the particle size (Zav=18.7-20.9 nm) after 1w/+4-RT-37° C. and 2 F/T cycles. The increase in particle size after 3 F/T cycles is more significant. CpG content=101 μg/ml. LS content <0.5 μg/ml

### Conclusions

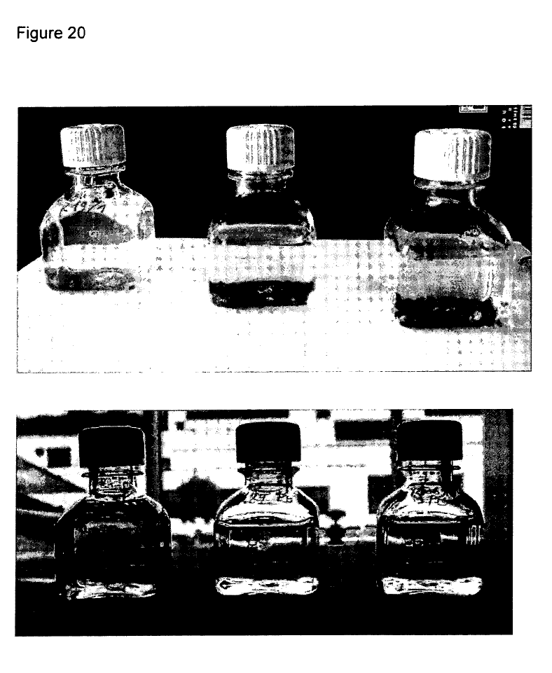

The R26/1 run indicates that a 100 μg/ml CpG spiking in HA-FT is appropriate for at the 1 L scale and addresses the precipitation of PRAME. (FIG. 20/21)

### Example 5

### Effect of PLG and Additional CpG Oligonucleotides on PRAME PB Solubility

### CPG and PLG Solutions Preparation and Quantification

Stock solutions of CpG 15-mers and 30-mers were prepared at 30 mg/ml in water (Stock solution of CpG-24mers already available at 30 mg/ml). Stock solution of PolyGlutamate (PLG)-24mers was prepared at 10 mg/ml in water. The three stock solutions were filtered on 0.22 μm PVDF membranes (millex GV). CpG content in the stock solutions was determined by RMN analysis. The content is 30.20 mg/ml for CpG 15-mers and 29.34 mg/ml for CpG 30-mers. PolyGlutamate (PLG)-24mers content was based on weighing.

### Dialysis Trials

A Dialysis step is proposed to remove the original solubilizing agent (Lauryl Sarcosyl—required to maintain PRAME solubility) and replace it by an alternative candidate under evaluation.

### Experimental Conditions:

Starting sample: 2 ml of PRAME Purified Bulk in 5 mM Borate-3.15% sucrose-300 ppm Lauryl Sarcosyl-pH 9.8 buffer.

According to experimental plan, some samples are spiked with candidate solubilizing agent (see Table 10)
Dialysis membrane cut-off: 20 kDa
Dialysis buffer: 2×1 L of 5 mM borate-3.15% sucrose-pH 9.8.
   According to experimental plan, some buffers are spiked with candidate solubilizing agent (see Table 10).
Each sample is dialysed against 1 L buffer under gentle agitation for 2 h at room temperature. Then the buffer is renewed and dialyse is pursued under gentle agitation overnight at room temperature.
Volumes of 2.0 ml to 2.1 ml for each sample were recovered after dialyse (negligible dilution effect).

TABLE 10
Dialysis trials

| | | | Sample |
|---|---|---|---|
| Sample | Sample spiking | Dialysis buffer | Reference |
| Purified Bulk in | No spiking | No dialysis (sample stored | A |
| 5 mM Borate buffer | | at −70° C.) | |
| pH 9.8 - 3.15% | No spiking | No dialysis (sample left | B |
| sucrose - 300 ppm | | overnight at RT) | |
| Sarcosyl | No spiking | 5 mM Borate buffer pH | C |
| | | 9.8 - 3.15% sucrose | |
| | Spiking with CpG15-mers | 5 mM Borate buffer pH | D |
| | solution up to 100 μg/ml | 9.8 - 3.15% sucrose - | |
| | | 100 μg/ml CpG 15-mers | |
| | Spiking with CpG24-mers | 5 mM Borate buffer pH | E |
| | solution up to 100 μg/ml | 9.8 - 3.15% sucrose - | |
| | | 100 μg/ml CpG 24-mers | |
| | Spiking with CpG30-mers | 5 mM Borate buffer pH | F |
| | solution up to 100 μg/ml | 9.8 - 3.15% sucrose - | |
| | | 100 μg/ml CpG 30-mers | |
| | Spiking with PLG-24mers | 5 mM Borate buffer pH | G |
| | solution up to 100 μg/ml | 9.8 - 3.15% sucrose - | |
| | | 100 μg/ml PLG 24-mers | |
| | Spiking with CpG15-mers | 5 mM Borate buffer pH | H |
| | solution up to 100 μg/ml | 9.8 - 3.15% sucrose | |
| | Spiking with CpG24-mers | | I |
| | solution up to 100 μg/ml | | |
| | Spiking with CpG30-mers | | J |
| | solution up to 100 μg/ml | | |
| | Spiking with PLG-24mers | | K |
| | solution up to 100 μg/ml | | |

### Analysis

### PRAME Content by RPC

PD1/3-Prame-His content is determined using a Reverse-Phase High Performance Liquid Chromatography system coupled with a UV detector. Standards and samples are diluted in the appropriate buffer prior to pre-treatment in Sodium Dodecyl Sulfate solution.

Detection of PD1/3-Prame-His is performed at 214 nm. Calibration curve is prepared with a PD1/3-Prame-His reference standard of known protein concentration. After plotting the PD1/3-Prame-His peaks areas in function of the concentration of standard solutions, the PD1/3-Prame-His content is deduced from the equation of the linear regression.

TABLE 11
PRAME content

| | | | Sample | |
|---|---|---|---|---|
| Sample | Sample spiking | Dialysis buffer | Reference | μg/ml |
| Purified Bulk in | No spiking | No dialysis (sample stored | A | 1806 |
| 5 mM Borate buffer | | at −70° C.) | | |
| pH 9.8 - 3.15% | No spiking | No dialysis (sample left | B | 1639 |
| sucrose - 300 ppm | | overnight at RT) | | |
| Sarcosyl | No spiking | 5 mM Borate buffer pH | C | 1649 |
| | | 9.8 - 3.15% sucrose | | |
| | Spiking with CpG15- | 5 mM Borate buffer pH | D | 1708 |
| | mers solution up to | 9.8 - 3.15% sucrose - | | |
| | 100 μg/ml | 100 μg/ml CpG 15-mers | | |
| | Spiking with CpG24- | 5 mM Borate buffer pH | E | 1650 |
| | mers solution up to | 9.8 - 3.15% sucrose - | | |
| | 100 μg/ml | 100 μg/ml CpG 24-mers | | |
| | Spiking with CpG30- | 5 mM Borate buffer pH | F | 1613 |
| | mers solution up to | 9.8 - 3.15% sucrose - | | |
| | 100 μg/ml | 100 μg/ml CpG 30-mers | | |
| | Spiking with PLG- | 5 mM Borate buffer pH | G | 1612 |
| | 24mers solution up to | 9.8 - 3.15% sucrose - | | |
| | 100 μg/ml | 100 μg/ml PLG 24-mers | | |
| | Spiking with CpG15- | 5 mM Borate buffer pH | H | 1692 |
| | mers solution up to | 9.8 - 3.15% sucrose | | |
| | 100 μg/ml | | | |
| | Spiking with CpG24- | | I | 1662 |
| | mers solution up to | | | |
| | 100 μg/ml | | | |
| | Spiking with CpG30- | | J | 1634 |
| | mers solution up to | | | |
| | 100 μg/ml | | | |
| | Spiking with PLG- | | K | 1616 |
| | 24mers solution up to | | | |
| | 100 μg/ml | | | |

The Prame content measured in all samples was consistent in all samples. No significant differences in Prame content could be observed between the sample in Borate buffer and the samples containing different meres CpG or PLG.

### Sarcosyl Content by RP-HPLC

A measure of the residual LS content is done after dialyse to ensure that the LS has been well removed.

### Material & Method: LS Content is Measured by RP-HPLC Technique

Column: Waters SunFire C18 5 μm (4.6×100 mm Column)
UV detector (214 nm)
Flow: 1 ml/min
Temperature: 40° C.
Elution gradient:
   Solvent A: 95% Acetonitrile-5% H2O-0.1% TFA
   Solvent B: 5% Acetonitrile-95% H2O-0.1% TFA

TABLE 12

| Phase | Time (min) | % A |
|---|---|---|
| Equilibration | 0 | 50 |
| Gradient | 1.4 | 100 |
| Step | 5.6 | 100 |
| Gradient | 5.9 | 50 |
| Step | 9.9 | 50 |

### Results

TABLE 13
LS content

| Sample | LS content |
|---|---|
| A | 268.0 |
| B | 267.5 |
| C | 4.0 |
| D | 5.0 |
| E | 1.9 |
| F | 3.0 |
| G | <0.5 |
| H | <0.5 |
| I | 2.4 |
| J | <0.5 |
| K | 2.8 |

We conclude to an efficient removal of LS for dialysed samples (C to K): all LS concentration measured ranges between <0.5 μg/ml and 5.0 μg/ml.

### CpG and PLG Content by IEX.

The amount of CpG and PLG in all samples was calculated by HPLC using homologous material as reference standard. It is demonstrated in the table below that the poly anions were present at a concentration close to 100 μg/ml as intended.

TABLE 14
PLG and CpG content

| | | | PLG 24 mers |
|---|---|---|---|
| PLG content | Diafiltration | Echantillons | (μg/mL) |
| spike 100 μg | contre buffer + CpG | G | 105 |
| PLG 24 mers | | | |
| spike 100 μg | contre buffer seul | K | 105 |
| PLG 24 mers | | | |
| | | | CpG |
| CpG content | Diafiltration | Echantillons | μg/ml |
| spike 100 μg | contre buffer + CpG | D | 98 |
| CpG 15 mers | | | |
| spike 100 μg | contre buffer seul | H | 100 |
| CpG 15 mers | | | |
| spike 100 μg | contre buffer + CpG | E | 99 |
| CpG 24 mers | | | |
| spike 100 μg | contre buffer seul | I | 95 |
| CpG 24 mers | | | |
| spike 100 μg | contre buffer + CpG | F | 108 |
| CpG 30 mers | | | |
| spike 100 μg | contre buffer seul | J | 107 |
| CpG 30 mers | | | |

### Size by Dynamic Light Scattering

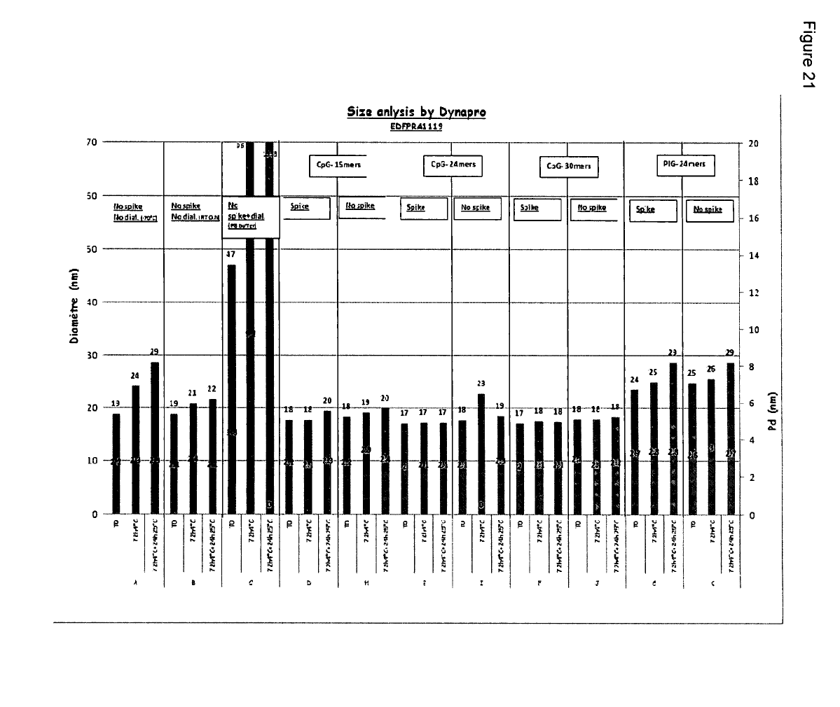

FIG. 21/21 demonstrates that antigen size is controlled in presence of CpG-15mers, CpG 24-mers, CpG 30-mers and PLG. CpG's seems to have a very slight better impact on antigen size stability than PLG (concentration improvement to consider).

1-28. (canceled)

29. A method for reducing the aggregation of a protein during a diluent exchange from diluent A to diluent B comprising:
(i) adding a polyanionic compound to diluent A prior to or contemporaneously with the exchange; and
(ii) exchanging protein from diluent A to diluent B, wherein the protein is PRAME.

30. The method according to claim 29, wherein the polyanionic compound is added prior to the diluent exchange.

31. The method or use according to claim 29, wherein diluent A comprises a detergent.

32. The method according to claim 31, wherein the detergent is an anionic detergent.

33. The method according to claim 32, wherein the detergent is selected from the group consisting of: SDS, sodium docusate and lauryl sarcosyl.

34. The method of claim 29, wherein diluent B is substantially free of detergent.

35. The method of claim 29, wherein diluent B comprises 5.0 mM borate, sucrose 3.15% w/v at pH 9.8.

36. The method of claim 29, wherein the protein comprises a His-tag.

37. The method of claim 29, wherein the polyanionic compound has a net negative charge of at least 8.

38. The method of claim 29, wherein the polyanionic compound is an oligonucleotide.

39. The method of claim 38, wherein the oligonucleotide is 5 to 200 nucleotides in length.

40. The method of claim 39, wherein the oligonucleotide comprises a CpG.

41. The method of claim 40, wherein the oligonucleotide is selected from the group consisting of:
SEQ ID NO: 1
TCC ATG ACG TTC CTG ACG TT (CpG 1826;) -,
SEQ ID NO: 2
TCT CCC AGC GTG CGC CAT (CpG 1758) -,
SEQ ID NO: 3
ACC GAT GAC GTC GCC GGT GAC GGC ACC ACG -,
SEQ ID NO: 4
TCG TCG TTT TGT CGT TTT GTC GTT
(CpG 2006/CpG7909),
SEQ ID NO: 5
TCC ATG ACG TTC CTG ATG CT (CpG 1668),
SEQ ID NO: 6
TCG ACG TTT TCG GCG CGC GCC G (CpG 5456),
SEQ ID NO: 9
TCG TCG TTT TGT CGT (CpG 15 mer):,
or
SEQ ID NO: 10
TCG TCG TTT TGT CGT TTT GTC GTT TCG TCG
(CpG 30 mer):.

42. The method of claim 41, wherein the diluent exchange is achieved by dialysis or diafiltration.

43. The method of claim 42, wherein the method further comprises step (iii) formulating the protein into diluent C.

44. The method of claim 43, wherein the diluent C comprises Tris, sucrose, borate, poloaxmer and CpG.

45. A composition comprising PRAME produced by the method of claim 44.

46. A composition comprising PRAME and an oligonucleotide, wherein PRAME has a particle size of between 10-30 nm.

47. The composition according to claim 46, wherein PRAME has a particle size of between 15-25 nm.

48. The composition according to claim 47, wherein the particle size is determined by dynamic light scattering.

49. A process for producing a pharmaceutically acceptable PRAME composition comprising the steps of:
(a) carrying out a diluent exchange according to the method of claim 29; and
(b) sterilising the formulation produced in step (a).

50. The process of claim 49 comprising an additional step (b′) formulating the protein into diluent C prior to step (b).

51. The process according to claim 50, comprising the additional step (c) lyophilising the formulation produced in step (b).

52. The process of claim 49, wherein the sterilisation is achieved by filtration.

53. A process for producing a pharmaceutically acceptable PRAME composition comprising the steps of:
(a) carrying out a diluent exchange of PRAME from diluent A to diluent B, wherein a polyanionic compound is added to diluent A or diluent B prior to or during the diluent exchange; and
(b) obtaining diluent B comprising PRAME.

54. The process of claim 53 comprising an additional step (c) selected from the group consisting of:
(i) sterilising the diluent B comprising PRAME; and
(ii) first formulating the diluent B comprising PRAME into diluent C comprising PRAME and second sterilising the diluent C comprising PRAME.

55. The process according to claim 53, comprising the additional step of lyophilising the PRAME composition.


SEQUENCE LISTING (10 sequences)

SEQ ID NO: 1 - DNA, 20 bp, Artificial Sequence
  Immunostimulant
tccatgacgttcctgacgtt

SEQ ID NO: 2 - DNA, 18 bp, Artificial Sequence
  Immunostimulatory oligonucleotide
tctcccagcgtgcgccat

SEQ ID NO: 3 - DNA, 30 bp, Artificial Sequence
  Immunostimulatory oligonucleotide
accgatgacgtcgccggtgacggcaccacg

SEQ ID NO: 4 - DNA, 24 bp, Artificial Sequence
  Immunostimulatory oligonucleotide
tcgtcgttttgtcgttttgtcgtt

SEQ ID NO: 5 - DNA, 20 bp, Artificial Sequence
  Immunostimulatory oligonucleotide
tccatgacgttcctgatgct

SEQ ID NO: 6 - DNA, 22 bp, Artificial Sequence
  Immunostimulatory oligonucleotide
tcgacgttttcggcgcgcgccg

SEQ ID NO: 7 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 8 - protein, 626 aa, Homo sapiens
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPNHHHHHH

SEQ ID NO: 9 - DNA, 15 bp, Artificial Sequence
  Immunostimulatory oligonucleotide
tcgtcgttttgtcgt

SEQ ID NO: 10 - DNA, 30 bp, Artificial Sequence
  Immunostimulatory oligonucleotide
tcgtcgttttgtcgttttgtcgtttcgtcg